(19) United States
(12) Patent Application Publication
(10) Pub. No.: US 2007/0197430 A1
(43) Pub. Date: Aug. 23, 2007

(54) Conjugates And Therapeutic Uses Thereof

(76) Inventors: Jonathan Baell, Ivanhoe (AU); Andrew Henry Wei, Surrey Hills (AU); Denis Bernard Scanlon, Elsternwick (AU)

Correspondence Address: PROSKAUER ROSE LLP, 1001 PENNSYLVANIA AVE, N.W.,, SUITE 400 SOUTH, WASHINGTON, DC, 20004, US

(21) Appl. No.: 11/571,160

(22) PCT Filed: Jun. 24, 2005
(86) PCT No.: PCT/AU05/00918

§ 371 Date: Mar. 22, 2007

Related U.S. Application Data
(60) Provisional application No. 60/582,398, filed on Jun. 24, 2004.

(51) Int. Cl. A61K 38/12; C07K 7/64

(52) U.S. Cl. 514/9; 530/317

(57) ABSTRACT

Conformationally constrained peptides that mimic BH3-only proteins and their conjugation to antibodies and other cell targeting compounds, compositions containing the conjugates and their use in the regulation of cell death are disclosed. The conformationally constrained peptides are capable of binding to and neutralising pro-survival Bcl-2 proteins. Processes for preparing the conformationally constrained peptides conjugated to antibodies and other cell targeting compounds and use of the conjugates in the treatment and/or prophylaxis of diseases or conditions associated with deregulation of cell death are also disclosed.

### FIELD OF THE INVENTION

This invention relates generally to conformationally constrained peptides that mimic BH3-only proteins and their conjugation to antibodies and other cell targeting compounds, to compositions containing them and to their use in the regulation of cell death. More particularly the invention relates to conformationally constrained peptides that mimic BH3-only proteins that are capable of binding to and neutralizing pro-survival Bcl-2 proteins and their conjugation to antibodies and other cell targeting compounds. The present invention also relates to processes of preparing the conformationally constrained peptides conjugated to antibodies and other cell targeting compounds and to their use in the treatment and/or prophylaxis of diseases or conditions associated with the deregulation of cell death.

### BACKGROUND OF THE INVENTION

Bibliographic details of various publications referred to in this specification are collected at the end of the description.

In the last decade, much has been learnt about the molecular control of programmed cell death (apoptosis), the evolutionary conserved process of killing and removing excess, unwanted or damaged cells during development and in tissue homeostasis. Since the deregulation of apoptosis has been linked to a number of disease states, our understanding of how this process is controlled may allow novel ways to treat diseases, either by promoting or by inhibiting apoptosis (Thompson, 1995). For example, loss of myocardial tissues after acute myocardial infarcts may be limited by inhibiting apoptosis in the damaged tissues. Excessive apoptosis is also a feature of neurodegenerative conditions such as Alzheimer's disease, suggesting that drugs preserving neuronal integrity may have a role in delaying the loss of vital neurons. In contrast to excess cell death, insufficient apoptosis is a feature of malignant disease and autoimmunity (Strasser et al, 1997). In either condition, persistence of damaged or unwanted cells that should normally be removed can contribute to disease.

In malignancies, mutations affecting cell death regulatory proteins or those that sense cellular damage have been described in various tumors. Bcl-2, the prototypic member of the Bcl-2 family of proteins, was first discovered as the result of the t(11;14) chromosomal translocation in human follicular B-cell lymphoma which results in its overexpression (Tsujimoto et. al., 1985; Cleary et. al., 1986). Overexpression of Bcl-2, which functions to inhibit apoptosis (Vaux et. al., 1988) or its functional homologs have also been reported in other tumors. However, mutations to proteins involved in sensing DNA damage are much more common in tumors. It is estimated that over half of human cancers have a mutation of the tumor suppressor protein, p53, or ones affecting this pathway (Lane, 1992). p53 is necessary to elicit the appropriate cellular responses (growth arrest, apoptosis) to most forms of DNA damage. Interestingly p53 kills cells mainly by a Bcl-2-dependent mechanism since Bcl-2 overexpression can block most cell deaths induced by p53 (Lowe et. al., 1993; Strasser et. al., 1994). Both clinical observations and experiments in mouse models suggest that inhibition of apoptosis (e.g. p53 mutations, overexpression of Bcl-2) (Strasser et. al., 1990; Adams et. al., 1992) greatly promote oncogenic transformation caused by mutations that promote cellular proliferation alone (e.g. overexpression of c-Myc, p21^{ras} mutations). Thus, reversing the process of tumorigenesis by promoting cell death, such as by activating p53 function or by inhibiting Bcl-2 function, may allow novel ways to complement our current treatments for malignancies. Furthermore, most of the cytotoxic treatments currently used to treat malignant diseases work partly by inducing the endogenous cell death machinery (Fisher, 1994), although this has been disputed by others (Brown and Wouters, 1999). For example, radiotherapy and many chemotherapeutic drugs activate apoptotic machinery indirectly by inducing DNA damage. Since the majority of tumors have mutations affecting the p53 pathway, forms of therapy that target the p53 pathway are significantly blunted because of the frequent loss of p53 function. The resistance of tumor cells to conventional agents provides further impetus to discovering novel cytotoxic drugs that act directly on the cell death machinery.

The effectors of cell death are cysteine proteases of the caspase family that cleave vital cellular substrates after aspartate residues (Thornberry, 1998). The caspases are synthesised as inactive zymogens and are only activated in response to cellular damage, thereby allowing exquisite control of apoptosis during normal tissue functioning in order to prevent inappropriate cell deaths. There are at least two distinct pathways to activate caspases in mammalian cells (Strasser et. al., 2000). Binding of cognate ligands to some members of the TNF receptor superfamily induce cell death by activating the initiator caspase, caspase-8/FLICE, which is recruited to form oligomers on the receptor by the adaptor protein FADD/MORT-1 (Ashkenazi and Dixit, 1998). Once activated, caspase-8 can cleave downstream effector caspases such as caspases-3, -6, and -7, thereby massively amplifying the process.

A second pathway to caspase activation is that controlled by the Bcl-2 family of proteins (Adams and Cory, 2001). Overexpression of Bcl-2 can block many forms of physiologically (e.g., developmentally programmed cell deaths, death due to growth factor deprivation) and experimentally applied damage signals (e.g., cellular stress, radiation, most chemotherapeutic drugs). Bcl-2 controls the activation of the initiator caspase, caspase-9, by the adaptor protein Apaf-1, but this does not appear to be the critical or the sole molecule regulated by Bcl-2 (Moriishi et. al., 1999; Conus et. al., 2000; Hausmann et. al., 2000; Haraguchi et. al., 2000; Marsden et. al., 2002). In the nematode C. elegans, the Bcl-2 homologue CED-9 functions by sequestering the activity of the adaptor protein CED-4 which is required to activate the caspase CED-3 (Spector et. al., 1997; Chinnaiyan et. al., 1997; Wu et. al., 1997; Yang et. al., 1998; Chen et. al., 2000). However, a true mammalian homologue of CED-4 has not been discovered to date. The machinery is also more complex in mammals which express a number of structural and functional homologues of Bcl-2, namely Bcl-x_{L}, Bcl-w, Mcl-1 and A1 (Adams and Cory, 1998) (Cory and Adams, 2002). These pro-survival proteins are structurally similar, generally containing four conserved Bcl-2 homology domains (BH1-4), as well as a C-terminal hydrophobic region, promoting cell survival until antagonised by a family of distantly related proteins, the BH3-only proteins (Baell J and Huang D C, 2002).

The BH3-only proteins are so-called because they share with each other, and with the other members of the Bcl-2 family of proteins, only the short BH3 domain (Huang and Strasser, 2000). This short domain forms an α-helical region, the hydrophobic face of which binds onto a hydrophobic surface cleft present on pro-survival Bcl-2 (Sattler et. al., 1997; Petros et. al., 2000). The BH3-only proteins probably function to sense cellular damage to critical cellular structures or metabolic processes, and are then unleashed to initiate cell death by binding to and neutralising Bcl-2 (Huang and Strasser, 2000; Bouillet et. al., 1999). Normally, the BH3-only proteins are kept inert by transcriptional or translational mechanisms, thereby preventing inappropriate cell deaths. Recently, two BH3-only proteins that are transcriptional targets of the tumour suppressor protein p53 have been described, namely Noxa (Oda et. al., 2000) and Puma/Bbc3 (Yu et. al., 2001; Nakano and Wousden, 2001; Han et. al., 2001). These proteins are thus good candidates to mediate cell death induced by p53 activation (Vousden, 2000). Some other BH3-only proteins are controlled instead by post-translational mechanisms. In particular, two are sequestered to the cell's cytoskeletal network, Bim to the microtubules and Bmf to the actin cytoskeleton (Puthalakath et. al., 1999; Puthalakath et. al., 2001). Damage signals that impinge upon these cytoskeletal structures will activate Bim or Bmf freeing them to bind to pro-survival Bcl-2 located on the cytoplasmic face of the outer mitochondrial membrane as well as those of the nucleus and endoplasmic reticulum.

Recently it has been shown that the killing by the BH3-only proteins is dependent on the activity of a third family of Bcl-2-related proteins, namely the Bax sub-family (Zong et. al., 2001; Cheng et. al., 2001). Although these proteins bear three of the four homology domains and are structurally very similar to the pro-survival proteins (Suzuki et al, 2001), Bax, Bak and Bok/Mtd function instead to promote cell death. Biochemically, damage signals cause these proteins to aggregate and such oligomers may function to cause damage to mitochondrial membranes (Eskes et. al., 2000; Desagher et. al., 1999; Antonsson et. al.; 2001; Mikhailov et. al., 2001; Wei et. al., 2001; Jürgensmeier et. al., 1998), thereby causing the release of mitochondrial pro-apoptogenic factors such as Smac/Diablo (Verhagen et. al., 2000; Du et. al., 2000) and cytochrome c, which is essential for the activation of caspase-9 by Apaf-1 (Kluck et. al., 1997; Yang et. al., 1997; Zou et. al., 1997; Li et. al., 1997). Since killing by BH3-only proteins depends on Bax and Bak in fibroblasts, their physiological role may be to activate Bax and Bak (Zong et. al., 2001; Korsmeyer et. al., 2000). In such a model, the pro-survival Bcl-2 proteins function merely to sequester the BH3-only proteins until such time as when there is insufficient capacity to do so. However, there are few reports of direct binding of the BH3-only proteins to Bax and Bak and even in the case of the BH3-only protein Bid appears weak (Eskes et. al., 2000; Wei et. al., 2001; Wang et al., 1996). To date there are no experiments to directly compare the binding of BH3-only proteins with pro-survival Bcl-2 and to pro-apoptotic Bax.

In addition to the tenuous biochemical evidence for the direct binding of BH3-only proteins to Bax-like proteins, careful analyses of the available genetic data also suggests that pro-survival Bcl-2 rather than pro-apoptotic Bax is the direct target of BH3-only proteins. In the nematode C. elegans, all the killing induced by the BH3-only protein EGL-1 is dependent on the ability of EGL-1 to bind to and neutralise nematode Bcl-2, CED-9 (Conradt et. al., 1998; Parrish et. al., 2000). The situation is somewhat more complex in mammals because of the functional redundancy in each class of proteins. Instead of a single BH3-only protein (EGL-1) and a single Bcl-2 homologue (CED-9), mammals express multiple proteins of each sub-class making direct comparison with the nematode difficult. Furthermore, nematodes do not appear to express Bax-like proteins. However, if the Bcl-2-like proteins function merely to sequester BH3-only proteins, then the amount of pro-survival Bcl-2-like proteins in any cell type must be limiting. It is therefore surprising that mice lacking a single allele of the bcl-2 (Veis et. al., 1993; Nakayama et. al., 1994; Kamada et. al., 1995), bcl-x (Motoyama et. al., 1995; Motoyama et. al., 1999) or bcl-w (Ross et. al., 1998; Print et. al., 1998) genes are normal whereas the homozygous knock-out mice all have striking phenotypes in the cell types where these genes play a crucial role. This suggests that the pro-survival Bcl-2-like proteins are not limiting; instead analysis of mice lacking the BH3-only protein Bim suggest that this class of proteins is limiting (Bouillet et. al., 1999; Bouillet et. al., 2001). Taken together, the available data would suggest that BH3-only proteins directly bind to Bcl-2 and it is by neutralising Bcl-2 that BH3-only proteins can activate Bax-like proteins.

Thus, agents that directly mimic the BH3-only proteins may induce cell death and may therefore be of value therapeutically. As Bcl-2 controls the critical point that determines a cell's fate, this class of proteins represent an attractive target for drug design. In particular, since many of the oncogenic mutations, such as those to p53, result in defects in sensing cellular damage that would normally result in cell death by a Bcl-2-dependent mechanism, directly targeting Bcl-2 and its homologs may circumvent such mutations. This may also permit an alternative route to overcome tumor resistance to current treatments.

One difficulty in providing compounds that bind directly with Bcl-2 proteins is that Bcl-2 proteins are not only present in persistent damaged or unwanted cells related to disease states such as malignant disease and autoimmunity, but also in normal healthy cells. In order to minimise the risk of apoptosis in healthy cells caused by compounds that bind to Bcl-2 proteins, it is desirable to target delivery of the compounds to specific unwanted cells.

The use of certain antibodies to target particular cell types is an active area of research, particularly where the antibody is conjugated to the cell active agent (Wang et. al., 1997; Goulet et. al., 1997; Sapra and Allen, 2002; Marks et. al., 2003; Deardon, 2002; Ludwig et. al., 2003; Uckun et. al., 1995). For example, CD19, as a pan B-cell antigen, is an ideal target for immunotoxin therapy of B-lineage leukemia and lymphomas (Wang et. al., 1997; Goulet et. al., 1997; Sapra and Allen, 2002; Marks et. al., 2003; Dearden, 2002). Various cytotoxic agents, such as genistein, ricin analogues, doxorubicin, and cytotoxic peptides have been conjugated to anti-CD19 antibodies (Wang et. al., 1997; Goulet et. al., 1997; Sapra and Allen, 2002; Marks et. al., 2003; Deardon, 2002; Uckun et. al., 1995), in order to target and kill B-cells and treat B-cell associated cancer.

A BH₃ peptide has been conjugated to luteinizing hormone-releasing hormone (LHRH) to target LHRH receptors, which are overexpressed in several cancer cell lines but are not expressed in healthy human visceral organs (Dharap and Minko, 2003).

### SUMMARY OF THE INVENTION

The present invention is predicated in part on the discovery that conformationally constrained peptides that mimic BH3-only proteins exhibit significant pro-apoptotic activity and have increased resistance to proteolysis compared to unconstrained linear peptides and such peptides can be conjugated to a cell targeting compound to allow direct delivery to unwanted or damaged cells. This discovery has been reduced to practice in novel compound/protein conjugates, in compositions containing them and in methods for their preparation and use, as described hereinafter.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers of steps. In a first aspect of the invention there is provided a conjugate comprising at least one cell targeting moiety and at least one conformationally constrained peptide moiety or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group, or represents the linkage between the conformationally constrained peptide moiety and the cell targeting moiety;
R′ is H, a C-terminal capping group, an oligopeptide optionally capped by a C-terminal capping group, or represents the linkage between the conformationally constrained peptide moiety and the cell targeting moiety; and
m and n are 0 or 1, provided that at least one of m and n is 1;
wherein a conformational constraint is provided by a linker (L) which tethers two amino acid residues, Zaa₁ and Zaa₂, in the sequence, and wherein the cell targeting moiety and the conformationally constrained peptide moiety or pharmaceutically acceptable salt or prodrug thereof are coupled through R or R′ or a functionalized amino acid side chain in the amino acid sequence (I).

As used herein, the term “conjugate” refers to a molecule composed of at least two moieties, at least one cell targeting moiety coupled to at least one conformationally constrained peptide moiety. Thus, at least two moieties are releasably coupled, preferably by a covalent bond, more preferably a covalent bond that is able to be hydrolysed under specific cellular conditions to release the conformationally constrained peptide within a damaged or unwanted cell at its site of action. Examples of suitable covalent bonds able to be hydrolysed intracellularly include disulfide bonds, ester bonds and amide bonds. The conformationally constrained peptide moiety or a spacer, which may be present between the cell targeting moiety and the conformationally constrained peptide moiety, may include an enzyme, for example, a protease, recognition sequence to provide hydrolysis of a bond under specific conditions thereby releasing the conformationally constrained peptide.

As used herein, the term “cell targeting moiety” refers to a moiety which is able to interact with a target molecule expressed by an unwanted or damaged cell, preferably on the cell surface. Preferably, the target molecule is overexpressed in the unwanted or damaged cell and is not expressed in healthy cells. Suitable cell targeting moieties include proteins and antigen-binding molecules, which interact with target molecules in the damaged or unwanted cells. Suitable cell targeting moieties include, but are not limited to, hormones such as luteinizing hormone-releasing hormone and cytokines such as VEGF and EGF, and antibodies such as CD19, CD20, CD22, CD79a, CD2, CD3, CD7, CD5, CD13, CD33 and CD138, or antibodies targeting receptors such as Erb1 (also called EGFR), Erb2 (also called HER2 and NEU), Erb3 and Erb4. In a preferred embodiment the cell targeting moiety is an antibody that targets B-cells, for example, CD19, CD20, CD22 and CD79a. The conjugate may include one cell targeting moiety and one conformationally constrained moiety, one cell targeting moiety and multiple conformationally constrained moieties, more than one cell targeting moiety and one conformationally constrained moiety or more than one cell targeting moiety and multiple conformationally constrained moieties. In some embodiments, the conjugate comprises one cell targeting moiety and between one and 100 conformationally constrained moieties, preferably one and 50, more preferably one and 20, most preferably 3 and 15. In other embodiments the conjugate may have more than one cell targeting moiety. The two or more cell targeting moieties may be the same or different. If the two or more cell targeting moieties are different, the conjugate may be used to target cells which express target molecules for each cell targeting moiety, thereby increasing cell specificity.

As used herein, the term “antigen-binding molecule” refers to a molecule that has binding affinity for a target antigen, and extends to immunoglobulins, immunoglobulin fragments and non-immunoglobulin derived protein frameworks that exhibit antigen-binding activity.

In some embodiments, the cell-targeting moiety is an antigen-binding molecule that is immuno-interactive with a target molecule, typically a cell surface protein (e.g., a receptor), expressed by a cell that is the subject of targeting. Reference herein to “immuno-interactive” includes reference to any interaction, reaction, or other form of association between molecules and in particular where one of the molecules is, or mimics, a component of the immune system.

The antigen-binding molecule may be selected from immunoglobulin molecules such as whole polyclonal antibodies and monoclonal antibodies as well as sub-immunoglobulin-sized antigen-binding molecules. Polyclonal antibodies may be prepared, for example, by injecting a target molecule of the invention into a production species, which may include mice or rabbits, to obtain polyclonal antisera. Methods of producing polyclonal antibodies are well known to those skilled in the art. Exemplary protocols which may be used are described for example in Coligan et. al., “Current Protocols In Immunology”, (John Wiley & Sons, Inc, 1991), and Ausubel et. al., “Current Protocols In Molecular Biology” (1994-1998), in particular Section III of Chapter 11.

In lieu of the polyclonal antisera obtained in the production species, monoclonal antibodies may be produced using the standard method as described, for example, by Köhler and Milstein, 1975, or by more recent modifications thereof as described, for example, in Coligan et. al., 1991, by immortalising spleen or other antibody-producing cells derived from a production species which has been inoculated with target molecule of the invention.

Suitable sub-immunoglobulin-sized antigen-binding molecules include, but are not restricted to, Fv, Fab, Fab′ and F(ab′)₂ immunoglobulin fragments. In some embodiments, the sub-immunoglobulin-sized antigen-binding molecule does not comprise the Fc portion of an immunoglobulin molecule.

In some embodiments, the sub-immunoglobulin antigen-binding molecule comprises a synthetic Fv fragment. Suitably, the synthetic Fv fragment is stabilised. Exemplary synthetic stabilised Fv fragments include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent.

ScFvs may be prepared, for example, in accordance with methods outlined in Krebber et. al., 1997. Alternatively, they may be prepared by methods described in U.S. Pat. No. 5,091,513, European Patent No 239,400 or the articles by Winter and Milstein, 1991 and Plückthun et. al., 1996, In Antibody engineering: A practical approach. 203-252.

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond therebetween. Suitable methods of producing dsFv are described for example in Glockshuber et. al. 1990, Reiter et. al. 1994a, Reiter et. al. 1994b, Reiter et. al. 1994c, Webber et. al. 1995.

Also contemplated as sub-immunoglobulin antigen binding molecules are single variable region domains (termed dAbs) as for example disclosed in Ward et. al. 1989, Hamers-Casterman et al 1993, Davies & Riechmann, 1994.

In other embodiments, the sub-immunoglobulin antigen-binding molecule is a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In still other embodiments, the sub-immunoglobulin antigen binding molecule comprises non-immunoglobulin derived, protein frameworks. For example, reference may be made to Ku & Schultz, 1995, which discloses a four-helix bundle protein cytochrome b562 having two loops randomised to create complementarity determining regions (CDRs), which have been selected for antigen binding.

In some embodiments, the sub-immunoglobulin antigen-binding molecule comprises a modifying moiety. In illustrative examples of this type, the modifying moiety modifies the effector function of the molecule. For instance, the modifying moiety may comprise a peptide for detection of the antigen-binding molecule, for example in an immunoassay. Alternatively, the modifying moiety may facilitate purification of the antigen-binding molecule. In this instance, the modifying moiety includes, but is not limited to, glutathione-S-transferase (GST), maltose binding protein (MBP) and hexahistidine (HIS₆), which are particularly useful for isolation of the antigen-binding molecule by affinity chromatography. For the purposes of purification by affinity chromatography, relevant matrices for affinity chromatography are glutathione-, amylose-, and nickel- or cobalt-conjugated resins respectively as is well known in the art.

The sub-immunoglobulin antigen binding molecule may be multivalent (i.e., having more than one antigen binding site). Such multivalent molecules may be specific for one or more antigens (e.g., two target molecules expressed by a targeted cell). Multivalent molecules of this type may be prepared by dimerization of two antibody fragments through a cysteinyl-containing peptide as, for example disclosed by Adams et. al., 1993 and Cumber et. al., 1992. Alternatively, dimerization may be facilitated by fusion of the antibody fragments to amphiphilic helices that naturally dimerize (Pack and Plückthun, 1992) or by use of domains (such as the leucine zippers jun and fos) that preferentially heterodimerize (Kostelny et. al., 1992). In other embodiments, the multivalent molecule comprises a multivalent single chain antibody (multi-scFv) comprising at least two scFvs linked together by a peptide linker. For example, non-covalently or covalently linked scFv dimers termed “diabodies” may be used in this regard. Multi-scFvs may be bispecific or greater depending on the number of scFvs employed having different antigen binding specificities. Multi-scFvs may be prepared for example by methods disclosed in U.S. Pat. No. 5,892,020.

As used herein, the term “conformationally constrained” refers the stabilization of a desired conformation, preferably a helical conformation, relative to other possible conformations by means of a linker which is covalently bound to two amino acid residues in the sequence. The conformational constraint also increases resistance to proteolysis compared to peptides lacking conformational constraint.

As used herein, the term “amino acid” refers to compounds having an amino group and a carboxylic acid group. An amino acid may be a naturally occurring amino acid or non-naturally occurring amino acid and may be a proteogenic amino acid or a non-proteogenic amino acid. The amino acids incorporated into the amino acid sequences of the present invention may be L-amino acids, D-amino acids, α-amino acid, β-amino acids, sugar amino acids and/or mixtures thereof.

Suitable naturally occurring proteogenic amino acids are shown in Table 1 together with their one letter and three letter codes.

TABLE 1

| Amino Acid | one letter code | three letter code |
|---|---|---|
| L-alanine | A | Ala |
| L-arginine | R | Arg |
| L-asparagine | N | Asn |
| L-aspartic acid | D | Asp |
| L-cysteine | C | Cys |
| L-glutamine | Q | Gln |
| L-glutamic acid | E | Glu |
| glycine | G | Gly |
| L-histidine | H | His |
| L-isoleucine. | I | Ile |
| L-leucine | L | Leu |
| L-lysine | K | Lys |
| L-methionine | M | Met |
| L-phenylalanine | F | Phe |
| L-proline | P | Pro |
| L-serine | S | Ser |
| L-threonine | T | Thr |
| L-tryptophan | W | Trp |
| L-tyrosine | Y | Tyr |
| L-valine | V | Val |

Suitable non-proteogenic or non-naturally occurring amino acids may be prepared by side chain modification or by total synthesis. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄. The amino group of lysine may also be derivatized by reaction with fatty acids, other amino acids or peptides or labeling groups by known methods of reacting amino groups with carboxylic acid groups.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulfhydryl groups may be modified by methods such as carboxymethylation with iodoacetic acid or iodoacetamide; performic acid oxidation to cysteic acid; formation of a mixed disulfides with other thiol compounds; reaction with maleimide, maleic anhydride or other substituted maleimide; formation of mercurial derivatives using 4-chloromercuribenzoate, 4-chloromercuriphenylsulfonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulfenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino-butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Examples of suitable non-proteogenic or non-naturally occurring amino acids contemplated herein is shown in Table 2.

TABLE 2

| Non-conventional amino acid | Code |
|---|---|
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisoleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl- -aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanin | Nmhphe |
| N-(N-(3,3-diphenylpropyl) | Nnbhe |
| carbamylmethyl)glycine | |

Suitable β-amino acids include, but are not limited to, L-β-homoalanine, L-β-homoarginine, L-β-homoasparagine, L-β-homoaspartic acid, L-β-homoglutamic acid, L-β-homoglutamine, L-β-homoisoleucine, L-β-homoleucine, L-β-homolysine, L-β-homomethionine, L-β-homophenylalanine, L-β-homoproline, L-β-homoserine, L-β-homothreonine, L-β-homotryptophan, L-β-homotyrosine, L-β-homovaline, 3-amino-phenylpropionic acid, 3-amino-chlorophenylbutyric acid, 3-amino-fluorophenylbutyric acid, 3-amino-bromophenyl butyric acid, 3-amino-nitrophenylbutyric acid, 3-amino-methylphenylbutyric acid, 3-amino-pentanoic acid, 2-amino-tetrahydroisoquinoline acetic acid, 3-amino-naphthyl-butyric acid, 3-amino-pentafluorophenyl-butyric acid, 3-amino-benzothienyl-butyric acid, 3-amino-dichlorophenyl-butyric acid, 3-amino-difluorophenyl-butyric acid, 3-amino-iodophenyl-butyric acid, 3-amino-trifluoromethylphenyl-butyric acid, 3-amino-cyanophenyl-butyric acid, 3-amino-thienyl-butyric acid, 3-amino-5-hexanoic acid, 3-amino-furyl-butyric acid, 3-amino-diphenyl-butyric acid, 3-amino-6-phenyl-5-hexanoic acid and 3-amino-hexynoic acid.

Sugar amino acids are sugar moieties containing at least one amino group as well as at least one carboxyl group. Sugar amino acids may be based on pyranose sugars or furanose sugars. Suitable sugar amino acids may have the amino and carboxylic acid groups attached to the same carbon atom, α-sugar amino acids, or attached to adjacent carbon atoms, β-sugar amino acids. Suitable sugar amino acids include but are not limited to

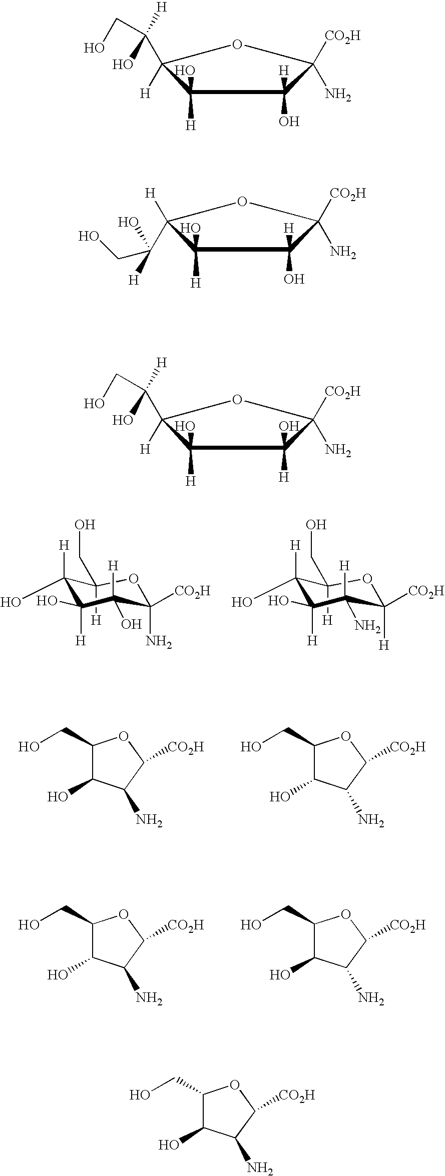

Sugar amino acids may be synthesized starting from commercially available monosaccharides, for example, glucose, glucosamine and galactose. The amino group may be introduced as an azide, cyanide or nitromethane group with subsequent reduction. The carboxylic acid group may be introduced directly as CO₂, by Wittig reaction with subsequent oxidation or by selective oxidation of a primary alcohol.

Haa₁, Haa₂, Haa₃ and Haa₄ are amino acids having hydrophobic side chains and provide the hydrophobic moieties for binding with the Bcl-2 protein. Haa₃ and at least two of Haa₁, Haa₂, and Haa₄ are required for binding. When one of Haa₁, Haa₂, and Haa₄ are not an amino acid having a hydrophobic side chain, they may be any amino acid as described for Xaa₁ below. Preferably all of Haa₁, Haa₂, Haa₃ and Haa₄ are amino acids having a hydrophobic side chain. Suitable Haa₁, Haa₂, Haa₃ and Haa₄ are selected from L-phenylalanine, L-isoleucine, L-leucine, L-valine, L-methionine, L-tyrosine, D-phenylalanine, D-isoleucine, D-leucine, D-valine, D-methionine, D-tyrosine, L-β-homophenylalanine, L-β-homoisoleucine, L-β-homoleucine, L-β-homovaline, L-β-homomethionine, L-β-homotyrosine, aminonorbornylcarboxylate, cyclohexylalanine, L-norleucine, L-norvaline, L-α-methylisoleucine, L-α-methylleucine, L-α-methylmethionine, L-α-methylnorvaline, L-α-methylphenylalanine, L-α-methylvaline, L-α-methyltyrosine, L-α-methylhomophenylalanine, D-α-methylleucine, D-α-methylmethionine, D-α-methylnorvaline, D-α-methylphenylalanine, D-α-methylvaline, D-α-methyltyrosine, D-α-methylhomophenylalanine residues L-tryptophan, L-3′4′-dichlorophenylalanine, L-1′-naphthylalanine and L-2′-naphthylalanine. Preferably Haa₁, Haa₂, Haa₃ and Haa₄ are independently selected from L-phenylalanine, L-isoleucine, L-leucine, L-valine, L-methionine and L-tyrosine. In a particularly preferred embodiment Haa₂ is L-leucine.

Saa is an amino acid residue having a small side chain. Suitable Saa residues include glycine, L-alanine, L-serine, L-cysteine, D-alanine, D-serine, D-cysteine, L-β-homoserine, L-β-homoalanine, γ-aminobutyric acid, aminoisobutyric acid, L-α-methylserine, L-α-methylalanine L-α-methylcysteine, D-α-methylserine, D-α-methylalanine and D-α-methylcysteine residues. Preferably Saa is selected from the group consisting of glycine, L-alanine, L-serine, L-cysteine and aminoisobutyric acid.

Naa is a negatively charged amino acid residue. Suitable Naa residues include L-aspartic acid, L-glutamic acid, D-aspartic acid, D-glutamic acid, L-β-homoaspartic acid, L-β-homoglutamic acid, L-α-methylaspartic acid, L-α-methylglutamic acid, D-α-methylaspartic acid and D-α-methylglutamic acid. Preferably Naa is an L-aspartic acid residue or an L-glutamic acid.

Xaa₁, Xaa₂, Xaa₃, Xaa₄ and Xaa₅ are independently selected from any amino acid as defined above and may be any naturally occurring, non-naturally occurring, proteogenic or non-proteogenic amino acid. Preferably Xaa₁, Xaa₂, Xaa₃, Xaa₄ and Xaa₅ are independently selected from L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine and L-valine. One or two of the residues Xaa₁, Xaa₂, Xaa₃, Xaa₄ and Xaa₅ may be Zaa₁ and Zaa₂ and provide the residues to which the linker (L) providing the conformational constraint is attached.

R is selected from H, an N-terminal capping group or an oligopeptide optionally capped by an N-terminal capping group. Preferably R is an N-terminal capping group or an oligopeptide having 1 to 10 amino acid residues selected from Xaa₁, optionally capped by an N-terminal capping group. Preferably the N-terminal capping group is a group that stabilises the terminus of a helix, usually having hydrogen atoms able to form hydrogen bonds or having a negative charge at the N-terminus to match with the helix dipole. Suitable N-terminal capping groups include acyl and N-succinate (HO₂CCH₂C(═O)) (Maison et. al., 2001). Alternatively, R represents the linkage of the conformationally constrained peptide to the cell targeting moiety, such as an antibody, either as a direct bond or through a spacer.

R′ is selected from H, a C-terminal capping group or an oligopeptide optionally capped by a C-terminal capping group. Preferably R′ is a C-terminal capping group or an oligopeptide having 1 to 10 amino acids selected from Xaa₁, optionally capped by a C-terminal capping group. Preferably the C-terminal capping group is a group that stabilises the terminus of a helix, usually having hydrogen atoms able to form hydrogen bonds or having a positive charge at the C-terminus to match with the helix dipole. A preferred C-terminal capping group is NH₂. Alternatively, R′ represents the linkage of the conformationally constrained peptide to the cell targeting moiety, such as an antibody, either as a direct bond or through a spacer.

The side chain of any amino acid in the conformationally constrained peptide moiety may be coupled, either directly or through a spacer, to the cell targeting moiety, provided that the amino acid has a suitably functionalized side chain and is not Zaa₁ or Zaa₂ or a residue required for binding to the Bcl-2 protein. The suitably functionalized side chain may be present in R or R′ when R or R′ are an oligopeptide. In some preferred embodiments, the amino acid which is coupled to the cell targeting moiety is Xaa₁, Xaa₃ or Xaa₄. Suitable amino acids that can be coupled to the cell targeting moiety through their side chains include, but are not limited to, lysine, cysteine, serine, aspartic acid, glutamic acid, homoaspartic acid, homoglutamic acid, homolysine, homoserine residue and the like. Preferably, the coupling side chain is on a lysine or cysteine residue.

When the functionalized side chain is linked to the cell targeting moiety through a spacer, the spacer may be from about 1 to about 100 atoms in length and may comprise one or more amino acid residues. The spacer may also incorporate moieties that assist in linkage between the constrained peptide and the cell targeting moiety, for example maleimide rings, an N-hydroxy succinimide activated form of maleimide, sulfosuccinimidyl-4-[N-maleimidomethyl]-cyclohexane-1-carboxylate or pyridyl sulfides, that were present on the cell targeting moiety to allow condensation with a cysteine residue or thiol group present on the constrained peptide or the spacer.

Furthermore, the cell targeting moiety may be linked to the constrained peptide, either directly or through a spacer, by way of a moiety incorporated into the constrained peptide to assist the peptide permeate through the cellular membrane. Examples of such moieties include fatty acids, short polyethylene glycols or a charged or polar amino acid sequence, such as -RRRRRRR- or -SSSS-, or a solubilizing sequence such as Sol. In this case, when the cell targeting moiety and the constrained peptide are cleaved at the site of action, the moiety that assists permeation through the cellular membrane remains intact.

The linker tethers two amino acid residues, Zaa₁ and Zaa₂, in the amino acid sequence. Preferably the linker tethers two non-adjacent amino acids that are suitably in an i(i+7) relationship where a first end of the linker is attached to a first amino acid residue (Zaa₁) at a first position in the sequence and the other end of the linker is attached to a second amino acid residue (Zaa₂) which appears in the sequence 7 amino acids after the first amino acid. Preferably the linker stabilizes a desired conformation, preferably a helical conformation. Preferably the linker has a length of 4 to 8 atoms and Zaa₁ and Zaa₂ are located in the amino acid sequence (i) in one of the following positions:
(a) i before Haa₁ at the N-terminal end of the amino acid sequence and
   i+7 between Haa₂ and Haa₃;
(b) i between Haa₁ and Haa₂ and
   i+7 between Haa₃ and Haa₄;
(c) i between Haa₂ and Haa₃ and
   i+7 after Haa₄ at the C-terminal end of the amino acid sequence.

In a preferred embodiment, the linker (L) is 4 to 8 atoms in length. The linker may be a hydrocarbon chain of 4 to 8 carbon atoms in length or one or more of the carbon atoms in the hydrocarbon chain may be replaced by a heteroatom selected from N, O or S. One or more of the atoms in the linker may be substituted with a substituent selected from ═O, OH, SH and CH₃. Alternatively, some of the carbon atoms may be replaced by a 1,4-disubstituted phenyl ring.

Zaa₁ and Zaa₂ may be any amino acid residue, however it is preferred that Zaa₁ and Zaa₂ are amino acid residues having side chains which are easily reacted with the linker precursor to form the linker. In a preferred embodiment, the linker covalently links two amino acid residues by the formation of amide bonds, that is, by forming a lactam bridge.

Preferably, Zaa₁ and Zaa₂ are independently selected from L-aspartic acid, L-glutamic acid, L-lysine, L-ornithine, D-aspartic acid, D-glutamic acid, D-lysine, D-ornithine, L-β-homoaspartic acid, L-β-homoglutamic acid, L-β-homolysine, L-α-methylaspartic acid, L-α-methylglutamic acid, L-α-methyllysine, L-α-methylomithine, D-α-methylaspartic acid, D-α-methylglutamic acid, D-α-methyllysine and L-α-methylomithine. Preferably, Zaa₁ and Zaa₂ are selected from L-aspartic acid, L-glutamic acid, L-lysine and L-ornithine. More preferably, Zaa₁ and Zaa₂ are selected from L-aspartic acid and L-glutamic acid.

When Zaa₁ and Zaa₂ have side chains containing a carboxylic acid, for example, L-aspartic acid or L-glutamic acid, preferred linkers are selected from the group consisting of —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)₂N⁺H₂(CH₂)₂NH—, —NH(CH₂)₂S(CH₂)₂NH—, —NHCH₂C(═O)NH(CH₂)₂NH—, —NH(CH₂)₂NHC(═O)CH₂NH—, —NH(CH₂)₂SS(CH₂)₂—NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(—O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH2)₃NHC(═O)(CH₂)₂NH—, —NH(CH₂)₃C(═O)NH(CH₂)₂NH— and —NH(CH₂)₂NHC(═O)(CH₂)₃NH—. More preferably the linker is selected from the group consisting of —NH(CH₂)₅NH—, —NH(CH₂)₆NH—, —NH(CH₂)₇NH—, —NHCH₂C(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—. Especially preferred linkers include —NH(CH₂)₅NH— and —NHCH₂C(═O)NH(CH₂)₂NH—.

When Zaa₁ and Zaa₂ have side chains containing an amino group, for example, L-lysine or L-ornithine, preferred linkers are selected from the group consisting of —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂—C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(—O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH₂)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—. More preferably the linker is selected from the group consisting of —C(═O)(CH₂)₅C(═O)—, —C(═O)(CH₂)₆C(═O)—, —C(═O)(CH₂)₇C(═O)—, —C(═O)CH₂C(═O)NH(CH₂)₂C(═O)—, —C(═O)(CH₂)₂NHC(═O)CH₂C(═O)—, —C(═O)(CH₂)₂O(CH₂)₃C(═O)— and —C(═O)(CH₂)₂C(═O)NH(CH₂)₂C(═O)—. Especially preferred linkers include —C(═O)(CH₂)₅C(═O)— and —C(═O)CH₂C(═O)NH(CH₂)₂C(═O)—.

When Zaa₁ has a side chain containing an amino group, for example, L-lysine or L-ornithine, and Zaa₂ has a side chain containing a carboxylic acid group, for example, L-aspartic acid or L-glutamic acid, preferred linkers are selected from the group consisting of —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂—NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(—O)(CH₂)₂C(═O)NH(CH₂)₃NH—, —C(═O)(CH2)₃NHC(═O)(CH₂)₂NH—, —C(═O)(CH₂)₃C(═O)NH(CH₂)₂NH— and —C(═O)(CH₂)₂NHC(—O)(CH₂)₃NH—. More preferably the linker is selected from the group consisting of —C(═O)(CH₂)₅NH—, —C(═O)(CH₂)₆NH—, —C(═O)(CH₂)₇NH—, —C(═O)CH₂C(═O)NH(CH₂)₂NH—, —C(═O)(CH₂)₂NHC(═O)CH₂NH—, —C(═O)(CH₂)₂O(CH₂)₃NH— and —C(═O)(CH₂)₂C(═O)NH(CH₂)₂NH—. Especially preferred linkers include —C(═O)(CH₂)₅NH— and —C(═O)CH₂C(═O)NH(CH₂)₂NH—.

When Zaa₁ has a side chain containing a carboxylic acid group, for example, L-aspartic acid or L-glutamic acid, and Zaa₂ has a side chain containing an amino group, for example, L-lysine or L-ornithine, preferred linkers are selected from the group consisting of —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)—, —NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)— and —NH(CH₂)₂NHC(═O)(CH₂)₃C(═O)—. More preferably the linker is selected from the group consisting of —NH(CH₂)₅C(═O)—, —NH(CH₂)₆C(═O)—, —NH(CH₂)₇C(═O)—, —NHCH₂C(═O)NH(CH₂)₂C(═O)—, —NH(CH₂)₂NHC(═O)CH₂C(═O)—, —NH(CH₂)₂O(CH₂)₃C(═O)— and —NH(CH₂)₂C(═O)NH(CH₂)₂C(═O)—. Especially preferred linkers include —NH(CH₂)₅C(═O)— and —NHCH₂C(═O)NH(CH₂)₂C(═O)—.

Preferably the amino acid sequence of the conformationally constrained peptide moiety is between 9 and 32 amino acid residues in length, more preferably between 9 and 31 amino acids in length, even more preferably between 9 and 30 amino acids in length, even more preferably between 9 and 29 amino acids in length, even more preferably between 9 and 28 amino acids in length, even more preferably between 9 and 27 amino acids in length, even more preferably between 9 and 26 amino acids in length, even more preferably between 9 and 25 amino acids in length, even more preferably between 9 and 24 amino acids in length, even more preferably between 9 and 23 amino acids in length, even more preferably between 9 and 22 amino acids in length, even more preferably between 9 and 21 amino acid residues in length, even more preferably between 9 and 20 amino acids in length, even more preferably between 9 and 19 amino acids in length, even more preferably between 9 and 18 amino acids in length, even more preferably between 9 and 17 amino acids in length, even more preferably 9 and 16 amino acid residues in length, even more preferably between 9 and 15 amino acids in length, even more preferably between 9 and 14 amino acids in length, and still even more preferably between 9 and 13 amino acids in length. An especially preferred amino acid sequence is between 9 and 12 amino acid residues in length.

Especially preferred conjugates of the invention comprise conformationally constrained peptide moieties as depicted in one of formulae (II) to (VI): 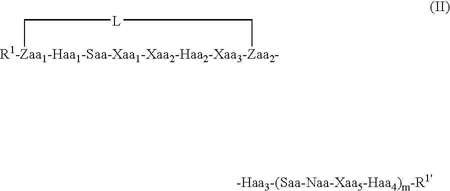
wherein Haa₁, Haa₂, Haa₃, Haa₄, Xaa₁, Xaa₂, Xaa₃, Xaa₅, Saa, Naa and L are as defined above for formula (I), m is 0 or 1, R¹ and R^{1′} are as defined above for R and R′ in formula (I), Zaa₁-L-Zaa₂ represents two amino acid residues with their side chains bridged by a linker L, and the cell targeting moiety is coupled to the peptide moiety through R¹, R^{1′} or through a functionalized amino acid side chain in the peptide; 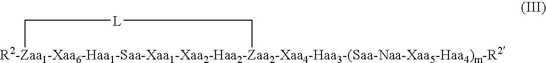
wherein Haa₁, Haa₂, Haa₃, Haa₄, Xaa₁, Xaa₂, Xaa₄, Xaa₅, Saa, Naa and L are as defined above for formula (I), Xaa₆ is an amino acid residue as defined for Xaa₁ above; m is 0 or 1, R² and R^{2′} are as defined above for R and R′ in formula (I), Zaa₁-L-Zaa₂ represents two amino acid residues with their side chains bridged by a linker L, and the cell targeting moiety is coupled to the peptide moiety through R², R^{2′} or through a functionalized amino acid side chain in the peptide; 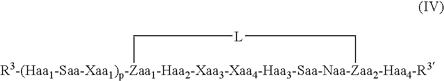
wherein Haa₁, Haa₂, Haa₃, Haa₄, Xaa₁, Xaa₃, Xaa₄, Saa, Naa and L are as defined above for formula (I), p is 0 or 1, R³ and R^{3′} are as defined above for R and R′ in formula (I), Zaa₁-L-Zaa₂ represents two amino acid residues with their side chains bridged by a linker L, and the cell targeting moiety is coupled to the peptide moiety through R³, R^{3′} or through a functionalized amino acid side chain in the peptide; 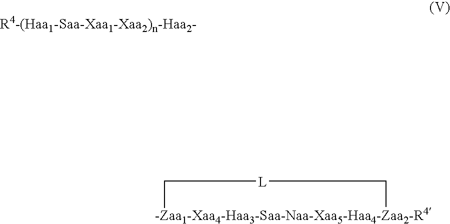
wherein Haa₁, Haa₂, Haa₃, Haa₄, Xaa₁, Xaa₂, Xaa₄, Xaa₅, Saa, Naa and L are as defined above in formula (I), n is 0 or 1, R⁴ and R^{4′} are as defined above for R and R′ in formula (I), Zaa₁-L-Zaa₂ represents two amino acid residues with their side chains bridged by a linker L, and the cell targeting moiety is coupled to the peptide moiety through R⁴, R^{4′} or through a functionalized amino acid side chain in the peptide; and 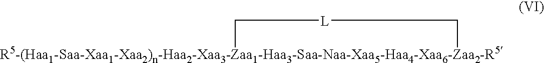
wherein Haa₁, Haa₂, Haa₃, Haa₄, Xaa₁, Xaa₂, Xaa₃, Xaa₅, Saa, Naa and L are as defined above for formula (I), Xaa₆ is an amino acid residue as defined for Xaa₁ above; n is 0 or 1, R⁵ and R^{5′} are as defined above for R and R′ in formula (I), Zaa₁-L-Zaa₂ represents two amino acid residues with their side chains bridged by a linker L, and the cell targeting moiety is coupled to the peptide moiety through R⁵, R^{5′} or through a functionalized amino acid side chain in the peptide; or a pharmaceutically acceptable salt or prodrug thereof.

Especially preferred conjugates of the invention include conformationally constrained peptide moieties derived from peptides of formula (VII): 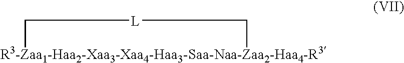
wherein Zaa₁, Haa₂, Xaa₃, Xaa₄, Haa₃, Saa, Naa, Zaa₂, Haa₄, R³, R^{3′} and L are defined above in formula (IV), and the cell targeting moiety is coupled to the peptide moiety through R³, R^{3′} or a functionalized amino acid side chain in the peptide.

Especially preferred conjugates of the invention include conformationally constrained peptide moieties derived from peptides of formula (VIII): 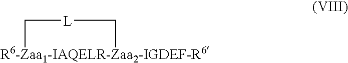
wherein R⁶ is Acetyl or represents a linkage with the cell targeting moiety,
R^{6′} is NH₂ or represents a linkage with the cell targeting moiety; and
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH— and —NH(CH₂)₂NHC(═O)(CH₂)₂NH—; or
where Zaa₁ and Zaa₂ are selected from L-lysine and ornithine; and
L is selected from —C(═O)(CH₂)₄C(═O)—, —C(═O)(CH₂)₅C(═O)—, —C(—O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)— and —C(═O)(CH₂)₂NHC(═O)(CH₂)₂C(═O)—; or
where Zaa₁ is selected from L-aspartic acid, L-glutamic acid and Zaa₂ is selected from L-lysine and ornithine; and
L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(—O)—, —NH(CH₂)₂SS(CH₂)₂C(—O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)— and —NH(CH₂)₂NHC(═O)(CH₂)₂C(═O)—; or
where Zaa₁ is selected from L-lysine and ornithine and Zaa₂ is selected from L-aspartic acid, L-glutamic acid; and
L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH— and —C(═O)(CH₂)₂NHC(═O)(CH₂)₂NH—;
and where the cell targeting moiety and the peptide moiety are coupled through R⁶, R^{6′} or a functionalized amino acid side chain in the peptide;
or conformationally constrained peptide moieties derived from peptides of formula (IX) 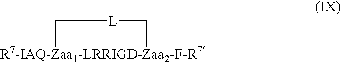
wherein R⁷ is Acetyl or represents a linkage with the cell targeting moiety;
R^{7′} is NH₂ or represents a linkage with the cell targeting moiety; and
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(—O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—; or
where Zaa₁ and Zaa₂ are selected from L-lysine and ornithine; and
L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(—O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(—O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH₂)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—; or
where Zaa₁ is selected from L-aspartic acid, L-glutamic acid and Zaa₂ is selected from L-lysine and ornithine; and
L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)—, —NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)— and —NH(CH₂)₂NHC(═O)(CH₂)₃C(═O)—; or
where Zaa₁ is selected from L-lysine and ornithine and Zaa₂ is selected from L-aspartic acid, L-glutamic acid; and
L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(—O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(═O)(CH₂)₂C(═O)NH(CH₂)₃NH—, —C(═O)(CH₂)₃NHC(═O)(CH₂)₂NH—, —C(═O)(CH₂)₃C(═O)NH(CH₂)₂NH— and —C(═O)(CH₂)₂NHC(═O)(CH₂)₃NH—,
and where the cell targeting moiety and the peptide moiety are coupled through R⁷, R^{7′} or a functionalized amino acid side chain in the peptide;
or conformationally constrained peptide moieties derived from peptides of any one of formulae (X) to (XVII): 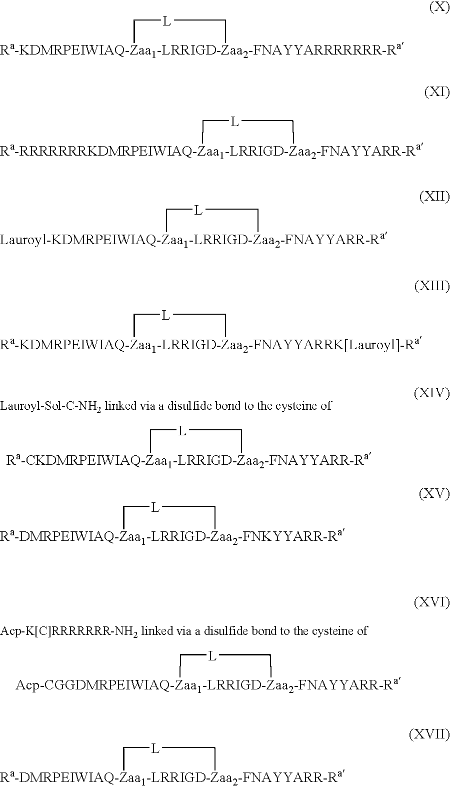
where R^{a} is acetyl or represents the linkage with the cell targeting moiety and R^{a′} is NH₂ or represents the linkage with the cell targeting moiety. In each case, the cell targeting moiety may be coupled to the peptide through R^{a}, R^{a′} or a functionalized amino acid side chain in the peptide, and
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH— and —NH(CH₂)₂NHC(═O)(CH₂)₂NH—; or
where Zaa₁ and Zaa₂ are selected from L-lysine and ornithine; and
L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)— and —C(═O)(CH₂)₂NHC(═O)(CH₂)₂C(═O)—; or
where Zaa₁ is selected from L-aspartic acid, L-glutamic acid and Zaa₂ is selected from L-lysine and ornithine; and
L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)— and —NH(CH₂)₂NHC(═O)(CH₂)₂C(═O)—; or
where Zaa₁ is selected from L-lysine and ornithine and Zaa₂ is selected from L-aspartic acid, L-glutamic acid; and
L is selected from —C(—O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH— and —C(═O)(CH₂)₂NHC(═O)(CH₂)₂NH—;
and wherein [C] represents a cysteine linked to a side chain such as a lysine side chain;
Lauroyl indicates that the fatty acid lauric acid is attached to the peptide either at the N-terminus or through a side chain such as lysine;
Acp indicates the inclusion of an aminocaproic acid spacer; and
Sol represents a solubilising sequence.

Preferred conformationally constrained peptide moieties derived from any one of formulae (X) to (XVII) are those in which Zaa₁ and Zaa₂ are glutamic acid and

L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH— and —NH(CH₂)₂NHC(═O)(CH₂)₂NH—.

Each of the conformationally constrained peptides of formulae (X) to (XVII) may optionally be linked to labels for use in assays. For example, the conformationally constrained peptides may be linked to a label such as fluoroscein isothiocyanate (Fitc), to determine internalisation of the peptides into cells, or biotin, to determine binding of the peptides to Bcl-2 proteins. Labels may be conveniently attached to a conformationally constrained peptide through a suitable amino acid side chain, such as lysine, through a spacer or the N- or C-terminus of the peptide. The amino acid residue carrying the amino acid side chain that may be linked to the label may be any amino acid residue in the sequence which is not bound to the conformational constraint or required for binding to the Bcl-2 protein. Suitable labels for use in assays include, but are not limited to, fluoroscein isothiocyanate (Fitc), rhodamine isothiocyanate (Ritc), tetramethyl rhodamine isothiocyanate (TRitc), fluoroscein dichlorotriazine (DTAF), phycoerythrin, phycocyanin, allophycocyanin, o-phthaldehyde, fluorescamine, biotin, streptavadin and the like. Other suitable labels are well known by those skilled in the art.

The conformationally constrained peptides of formulae (X) to (XIV) and (XVI) include a fatty acid, lauric acid or the sequence -RRRRRRR- to assist the peptides permeate through the cellular membrane. Any fatty acid may be attached to the conformationally constrained peptides to assist permeation through the cellular membrane. Preferred fatty acid esters include lauroyl, caproyl, myristoyl and palmitoyl. The solubilising sequence, Sol, may be present to assist with solubility. Suitable solubilising sequences are known in the art and include, but are not limited to, any charged or polar amino acid sequence containing one or more residues, such as -SSSS- or other polar or charged moieties, such as short polyethylene glycols (PEGs).

The use of peptides with disulfide linkages between a cell permeating fatty acid or sequences may be suitable prodrugs since after internalisation, the disulfide bonds may be cleaved inside the cell under reducing conditions.

Examples of especially preferred conformationally constrained peptide moieties include: 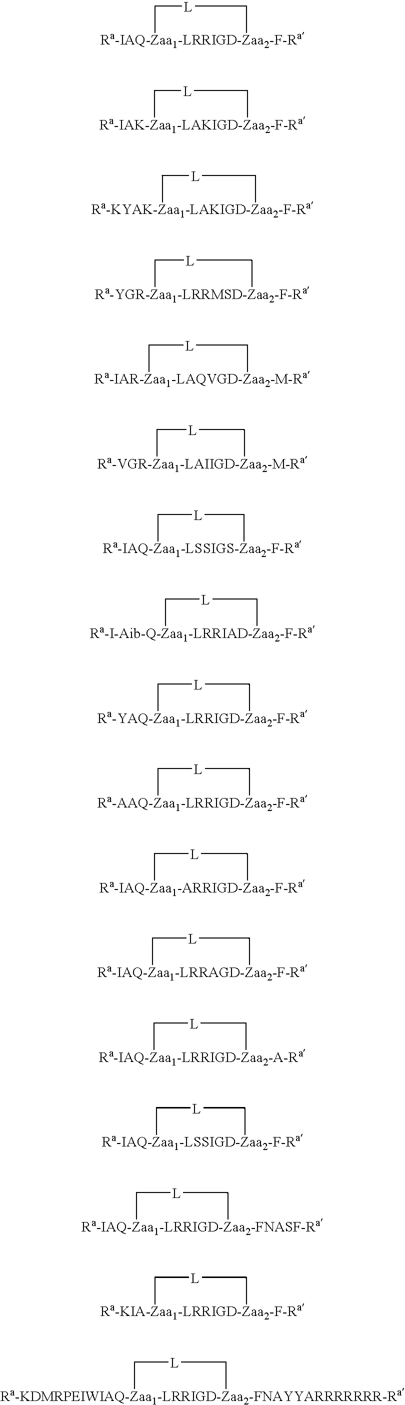 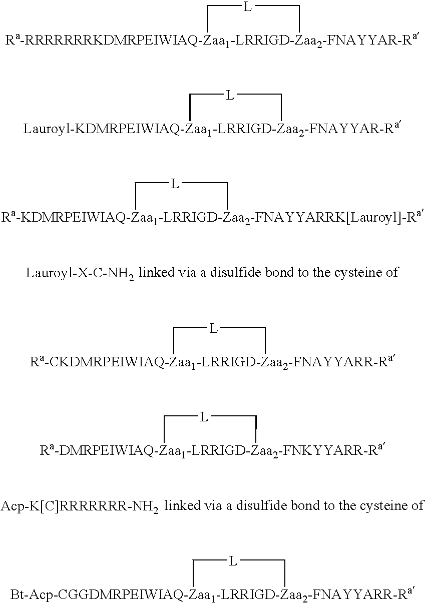
where R^{a} is Acetyl or represents a linkage to the cell targeting moiety, R^{a′} is NH₂ or represents a linkage to the cell targeting moiety and where the cell targeting moiety is coupled to the peptide through R^{a}, R^{a′} or a functionalized amino acid side chain in the peptide, and
where Zaa₁, Zaa₂ and L are as defined above. Preferably Zaa₁ and Zaa₂ are independently selected from L-aspartic acid and L-glutamic acid and preferably L is selected from —NH(CH₂)₅NH—, —NH(CH₂)₆NH—, —NH(CH₂)₇NH—, —NHCH₂(═O)NH(CH₂)₂NH—, —NH(CH₂)₂NHC(—O)CH₂NH—, —NH(CH₂)₂O(CH₂)₃NH— and —NH(CH₂)₂C(═O)NH(CH₂)₂NH—. Especially preferred linkers include —NH(CH₂)₅NH— and —NHCH₂C(═O)NH(CH₂)₂NH—.

Especially preferred conformationally constrained peptide moieties include: 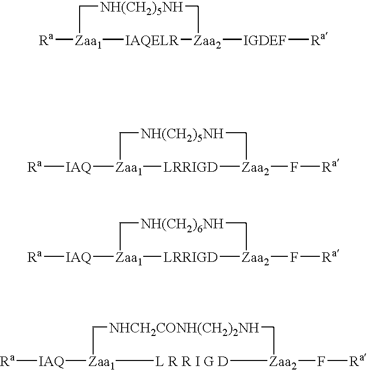
where R^{a} is Acetyl or represents a linkage to the cell targeting moiety, R^{a′} is NH₂ or represents a linkage to the cell targeting moiety and where the cell targeting moiety is coupled to the peptide through R^{a}, R^{a′} or a functionalized amino acid side chain in the peptide, and
where Zaa₁ and Zaa₂ are independently selected from L-aspartic acid and L-glutamic acid, especially L-glutamic acid.

The present invention also encompasses retro-inverso amino acid sequences in the conformationally constrained peptide moiety. The term “retro-inverso amino acid sequence” refers to an isomer of a linear peptide in which the direction of the sequence is reversed (“retro”) and the chirality of each amino acid residue is inverted (“inverso”), Jameson et al., 1994, Brady et al., 1994. For example, if the parent peptide is Thr-Ala-Tyr, the retro modified form is Tyr-Ala-Thr, the inverso modified form is thr-ala-tyr, and the retro-inverso form is tyr-ala-thr (lower case letters refer to D-amino acids). Compared to the parent peptide, a helical retro-inverso peptide can substantially retain the original spatial conformation of the side chains but has reversed peptide bonds, resulting in a retro-inverso isomer with a topology that closely resembles the parent peptide, since all peptide backbone hydrogen bond interactions are involved in maintaining the helical structure.

The conformationally constrained peptide moieties for use in the conjugates of the invention may be prepared using techniques known in the art. For example, peptides can be synthesized using various solid phase techniques (See Roberge et. al.; 1995) or using an automated synthesis, for example, using a Pioneer peptide synthesizer and standard F-moc chemistry, Fields (1991).

The linear peptides can also be prepared using recombinant DNA techniques known in the art. For example, nucleotide sequences encoding a peptide having the required amino acid sequence, can be inserted into a suitable DNA vector, such as a plasmid. Techniques suitable for preparing a DNA vector are described in Sambrook, J., et. al., 1989. Once inserted, the vector is used to transform a suitable host. The recombinant peptide is then produced in the host by expression. The transformed host can be either a prokaryotic or eukaryotic cell.

Once the peptides have been prepared, they may be substantially purified by preparative HPLC. The composition of the synthetic peptides can be confirmed by amino acid analysis or by sequencing (using the Edman degradation procedure).

Alternatively, a nucleotide sequence encoding amino acid residues 88 to 99 of the Bim protein (relative to the full length Bim protein) can be mutagenised, for example, treated with a chemical mutagen, such as a base analog, a deaminating agent, or an alkylating agent, or with a physical mutagen, such as UV or ionizing radiation or heat, using techniques known in the art. The mutant nucleotide sequence can then be expressed in a suitable host and the recombinant polypeptide purified using standard protocols known to a person skilled in the art.

The linker may be incorporated into the peptide to form a conformationally constrained peptide moiety using known techniques. For example, when Zaa₁ and Zaa₂ are residues having an acidic side chain, such as aspartic acid or glutamic acid, each of Zaa₁ and Zaa₂ is selectively protected before the peptide is synthesised. After peptide synthesis, one of the protecting groups (P₁) is selectively removed and the resulting carboxylic acid group is reacted with the amine of the linker to form an amide bond. The other protecting group (P₂) is removed and the second carboxylic acid is reacted with another amine on the linker to form a second amide bond. This process is shown in Scheme 1.

Similarly, when Zaa₁ and Zaa₂ are residues having an amino side chain, such as lysine or ornithine, these residues may be reacted with a dicarboxylic acid. One of the amino groups on the amino acid side chain may be selectively protected before the peptide is synthesized. During the reaction, one of the carboxylic acid groups on the dicarboxylic acid linker precursor is selectively protected. The remaining carboxylic acid is reacted with the amine of the lysine or ornithine residue to form an amide bond. The carboxylic acid protecting group (P₁) and the amino protecting group (P₂) are removed and the second carboxylic acid is reacted with a second amine on a lysine or ornithine residue to form a second amide bond. This process is shown in Scheme 2.

A similar process may be used when one of Zaa₁ and Zaa₂ has an acidic side chain and the other has an amino side chain and the linker has one amino group and one carboxylic acid group. The linker can be incorporated by selective deprotection of one side chain, reaction with the linker, then deprotection of the other side chain and the remaining reactive group of the linker.

Suitable protecting and deprotecting methods for reactive functional groups such as carboxylic acids and amines are known in the art, for example, in Protective Groups in Organic Synthesis, T. W. Green & P. Wutz, John Wiley & Son, 3^{rd} Ed, 1999.

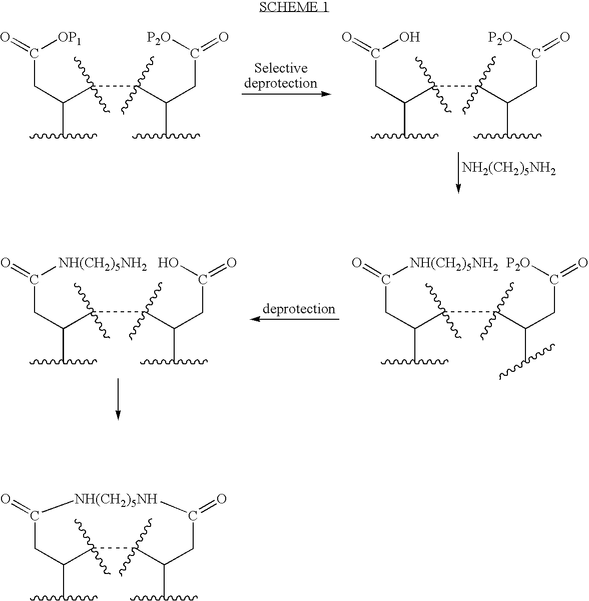

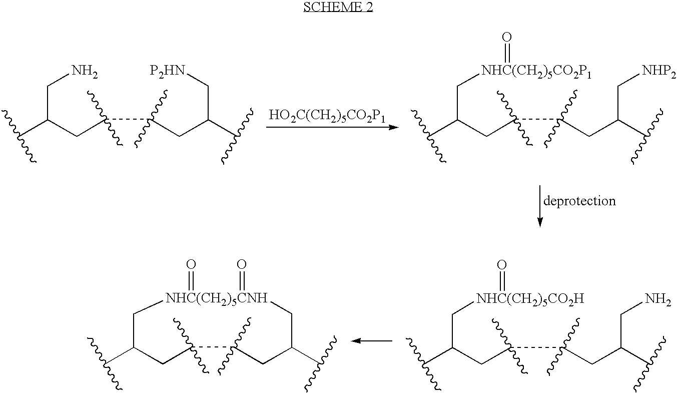

In an alternative synthesis, the linker is reacted with the side chain of the amino acid residue Z₁ or Z₂ before it is incorporated into the peptide. For example, if Z₁ and Z₂ are amino acids having a carboxylic acid in their side chain, for example aspartic acid or glutamic acid, and the linker is a diamino containing group, such as a diaminoalkyl group or another diamino group which would provide L as described above, the linker may be reacted with Z₁ or Z₂ before peptide synthesis occurs. For example, standard amide formation techniques may be used. An exemplary synthesis is shown in Scheme 3.

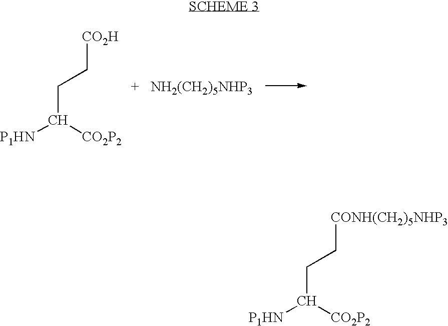

If Z₁ and Z₂ have amino acid side chains having an amino group in their side chain, for example lysine or ornithine, and the linker is a dicarboxylic acid group, such as an alkyldicarboxylic acid group or another dicarboxylic acid group that would provide L as described above, the linker may be introduced using standard amide formation techniques. An exemplary synthesis is shown in Scheme 4.

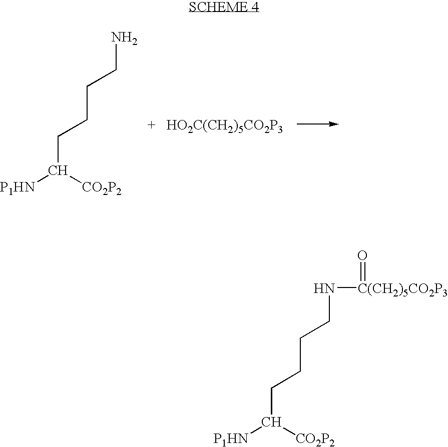

Similarly, if one of Z₁ and Z₂ is an amino acid having an amine containing side chain and the other has a carboxylic acid containing side chain and the linker is an amino carboxylic acid, the linker may be attached to Z₁ or Z₂ using standard amide formation techniques as described above. Exemplary syntheses are shown in Schemes 5 and 6.

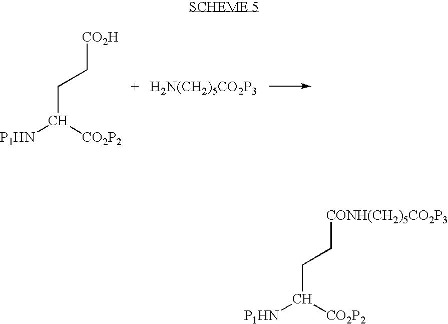

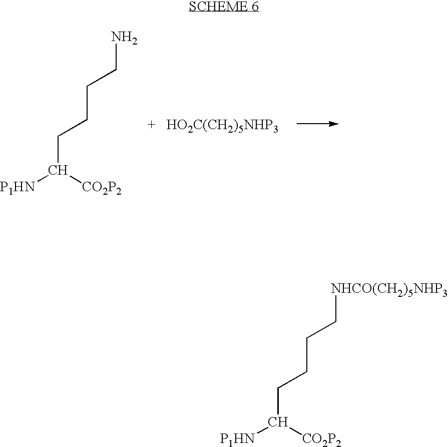

In Schemes 3-6, P₁, P₂ and P₃ are suitable protecting groups. P₃ may be present during coupling with the amino acid or may be introduced after coupling is complete. P₂ is preferably readily removable in the presence of P₁ to allow direct use in solid phase peptide synthesis. Preferably P₁ is Fmoc.

Once the amino acid coupled to the linker has been prepared, it may be incorporated into a peptide using standard peptide synthesis as described above, for example, solid phase synthesis or solution phase synthesis. After the peptide synthesis is complete, the protecting groups on the linker and on the amino acid residue, Z₁ or Z₂, which is not coupled to the linker are removed and the linker is then coupled to the second amino acid in the peptide by standard amide formation techniques. The coupling of the linker may be achieved while the peptide is still attached to the resin during solid phase synthesis or may be achieved after cleavage from the resin, in a solution phase.

An exemplary synthesis is shown in Scheme 7.

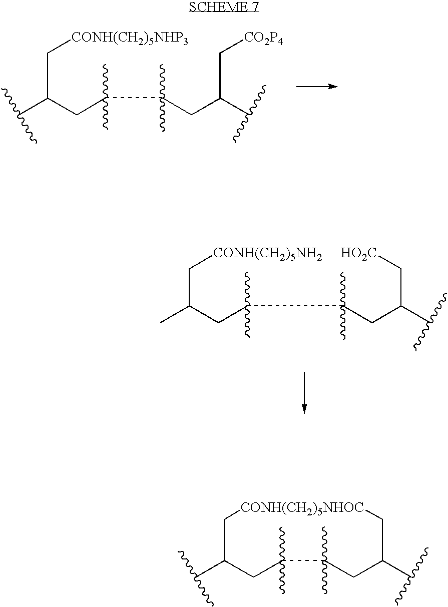

In preferred embodiments, the protecting groups used on the linker terminus and the side chain to which the linker is to be coupled are able to be selectively removed without removing other amino acid side chain protection in the peptide, before coupling of the linker terminus to the amino acid side chain occurs. Suitable protecting groups are readily determined by those working in peptide synthesis.

In preferred embodiments Z₁ and Z₂ are glutamic acid residues and one of the glutamic acids is coupled with a diaminoalkane such as 1,4-diaminobutane, 1,5-diaminopentane or 1,6-diaminohexane before synthesis of a peptide.

This alternative synthesis described in Schemes 3 to 7 is particularly useful in reducing or eliminating unwanted side reactions that occur during introduction of the linker.

It has also been found that during peptide synthesis, when the peptide has an aspartic acid residue adjacent to a glycine residue, unwanted aspartimide derivatives may be formed. This may be avoided by avoiding the use of benzyl protecting groups and using an Fmoc deprotection step with a solution of 0.2M HOBt/25% piperidine-DMF for 1 minute.

According to one aspect of the invention, there is provided a method of preparing a conformationally constrained peptide comprising the steps of:
(i) reacting a linker containing a first functional group and a second functional group with a reactive group on an amino acid side chain so that the first functional group of the linker is covalently coupled with the reactive group of the amino acid side chain;
(ii) protecting the second functional group of the linker if required;
(iii) incorporating the amino acid from (i) or (ii) into a peptide, said peptide comprising a second amino acid having a reactive side chain capable of covalently coupling with the second functional group of the linker;
(iv) deprotecting the second functional group of the linker if required; and
(v) reacting the second functional group of the linker with the reactive side chain of the second amino acid.

According to another aspect of the invention, there is provided a method according to the invention comprising the steps of:
(i) reacting a linker having one amino group and one optionally protected amino group or one amino group and one optionally protected carboxylic acid group, with an amino acid having a side chain comprising a carboxylic acid so that the linker and the amino acid side chain are coupled by an amide bond;
(ii) incorporating the amino acid from (i) into a peptide, said peptide comprising a second amino acid residue having a side chain capable of reacting with the uncoupled amino group or carboxylic acid group of the linker;
(iii) deprotecting the amino group or carboxylic acid group of the linker if required; and
(iv) reacting the second amino acid side chain with the amino group or carboxylic acid group of the linker to form an amide bond.

According to yet another aspect of the invention, there is provided a method according to the invention comprising the steps of:
(i) reacting a linker having one carboxylic acid group and one optionally protected carboxylic acid group or one carboxylic acid group and one optionally protected amino group, with an amino acid having a side chain comprising an amino group so that the linker and the amino acid side chain are coupled by an amide bond;
(ii) incorporating the amino acid from (i) into a peptide, said peptide comprising a second amino acid residue having a side chain capable of reacting with the uncoupled amino group or carboxylic acid group of the linker;
(iii) deprotecting the amino group or carboxylic acid group of the linker; and
(iv) reacting the second amino acid side chain with the carboxylic acid group or amino group of the linker to form an amide bond.

In some embodiments of the method, the amino acid residue in step (i) has a carboxylic acid group in its side chain, for example L-aspartic acid, L-glutamic acid, D-aspartic acid or D-glutamic acid, and the linker is selected from H₂N(CH₂)₄NH₂, H₂N(CH₂)₅NH₂, H₂N(CH₂)₆NH₂, H₂N(CH₂)₇NH₂, H₂N(CH₂)₂O(CH₂)₂NH₂, H₂N(CH₂)₂N⁺H₂(CH₂)₂NH₂, H₂N(CH₂)₂S(CH₂)₂NH₂, H₂NCH₂C(═O)NH(CH₂)₂NH₂, H₂N(CH₂)₂NHC(═O)CH₂NH₂, H₂N(CH₂)₂SS(CH₂)₂—NH₂, H₂N(CH₂)₂O(CH₂)₃NH₂, H₂N(CH₂)₂N⁺H₂(CH₂)₃NH₂, H₂N(CH₂)₂S(CH₂)₃NH₂, H₂N(CH₂)₂C(═O)NH(CH₂)₂NH₂, H₂N(CH₂)₂NHC(═O)(CH₂)₂NH₂, H₂NCH₂C(═O)NH(CH₂)₃NH₂, H₂N(CH₂)₃NHC(═O)CH₂NH₂, H₂NCH₂C(═O)NH(CH₂)₄NH₂, H₂N(CH₂)₄NHC(═O)CH₂NH₂, H₂N(CH₂)₂C(═O)NH(CH₂)₃NH₂, H₂N(CH2)₃NHC(═O)(CH₂)₂NH₂, H₂N(CH₂)₃C(═O)NH(CH₂)₂NH₂ and H₂N(CH₂)₂NHC(═O)(CH₂)₃NH₂. More preferably the linker is selected from the group consisting of H₂N(CH₂)₅NH₂, H₂N(CH₂)₆NH₂, H₂N(CH₂)₇NH₂, H₂NCH₂C(═O)NH(CH₂)₂NH₂, H₂N(CH₂)₂NHC(═O)CH₂NH₂, H₂N(CH₂)₂O(CH₂)₃NH₂ and H₂N(CH₂)₂C(═O)NH(CH₂)₂NH₂. Especially preferred linkers include H₂N(CH₂)₅NH₂ and H₂NCH₂C(═O)NH(CH₂)₂NH₂.

In some embodiments of the method, the amino acid residue in step (i) has an amino group in its side chain, for example L-lysine, ornithine or D-lysine, and the linker is selected from HOC(═O)(CH₂)₄C(═O)OH, HOC(═O)(CH₂)₅C(═O)OH, HOC(═O)(CH₂)₆C(═O)OH, HOC(═O)(CH₂)₇C(═O)OH, HOC(═O)(CH₂)₂O(CH₂)₂C(═O)OH, HOC(═O)(CH₂)N⁺H₂(CH₂)₂C(═O)OH, HOC(═O)(CH₂)S(CH₂)₂C(═O)OH, HOC(═O)CH₂C(═O)NH(CH₂)₂C(═O)OH, HOC(═O)(CH₂)₂NHC(═O)CH₂C(═O)OH, HOC(═O)(CH₂)₂SS(CH₂)₂—C(═O)OH, HOC(═O)(CH₂)₂O(CH₂)₃C(═O)OH, HOC(═O)(CH₂)₂N⁺H₂(CH₂)₃C(═O)OH, HOC(═O)(CH₂)₂S(CH₂)₃C(═O)OH, HOC(═O)(CH₂)₂C(═O)NH(CH₂)₂C(═O)OH, HOC(—O)(CH₂)₂NHC(═O)(CH₂)₂C(═O)OH, HOC(═O)CH₂C(═O)NH(CH₂)₃C(═O)OH, HOC(═O)(CH₂)₃NHC(═O)CH₂C(═O)OH, HOC(═O)CH₂C(═O)NH(CH₂)₄C(═O)OH, HOC(—O)(CH₂)₄NHC(═O)CH₂C(—O)OH, HOC(═O)(CH₂)₂C(═O)NH(CH₂)₃C(═O)OH, HOC(═O)(CH2)₃NHC(═O)(CH₂)₂C(═O)OH, HOC(═O)(CH₂)₃C(═O)NH(CH₂)₂C(═O)OH and HOC(═O)(CH₂)₂NHC(═O)(CH₂)₃C(═O)OH. More preferably the linker is selected from the group consisting of HOC(═O)(CH₂)₅C(═O)OH, HOC(═O)(CH₂)₆C(═O)OH, HOC(═O)(CH₂)₇C(═O)OH, HOC(═O)CH₂C(═O)NH(CH₂)₂C(═O)OH, HOC(═O)(CH₂)₂NHC(═O)CH₂C(═O)OH, HOC(═O)(CH₂)₂O(CH₂)₃C(═O)OH and HOC(═O)(CH₂)₂C(═O)NH(CH₂)₂C(═O)OH. Especially preferred linkers include HOC(═O)(CH₂)₅C(═O)OH and HOC(═O)CH₂C(═O)NH(CH₂)₂C(═O)OH.

In some embodiments of the method, the amino acid residue in step (i) has a carboxylic acid group or an amino group in its side chain, for example L-aspartic acid, L-glutamic acid, D-aspartic acid, D-glutamic acid, L-lysine, ornithine or D-lysine, and the linker is selected from HOC(═O)(CH₂)₄NH₂, HOC(═O)(CH₂)₅NH₂, HOC(═O)(CH₂)₆NH₂, HOC(═O)(CH₂)₇NH₂, HOC(═O)(CH₂)₂O(CH₂)₂NH₂, HOC(═O)(CH₂)N⁺H₂(CH₂)₂NH₂, HOC(═O)(CH₂)S(CH₂)₂NH₂, HOC(═O)CH₂C(═O)NH(CH₂)₂NH₂, HOC(═O)(CH₂)₂NHC(═O)CH₂NH₂, HOC(═O)(CH₂)₂SS(CH₂)₂—NH₂, HOC(═O)(CH₂)₂O(CH₂)₃NH₂, HOC(═O)(CH₂)₂N⁺H₂(CH₂)₃NH₂, HOC(═O)(CH₂)₂S(CH₂)₃NH₂. HOC(═O)(CH₂)₂C(═O)NH(CH₂)₂NH₂, HOC(═O)(CH₂)₂NHC(═O)(CH₂)₂NH₂, HOC(═O)CH₂C(═O)NH(CH₂)₃NH₂, HOC(═O)(CH₂)₃NHC(═O)CH₂NH₂, HOC(═O)CH₂C(═O)NH(CH₂)₄NH₂, HOC(═O)(CH₂)₄NHC(═O)CH₂NH₂, HOC(═O)(CH₂)₂C(═O)NH(CH₂)₃NH₂, HOC(═O)(CH2)₃NHC(═O)(CH₂)₂NH₂, HOC(═O)(CH₂)₃C(═O)NH(CH₂)₂NH₂ and HOC(═O)(CH₂)₂NHC(═O)(CH₂)₃NH₂. More preferably the linker is selected from the group consisting of HOC(═O)(CH₂)₅NH₂, HOC(═O)(CH₂)₆NH₂, HOC(═O)(CH₂)₇NH₂, HOC(═O)CH₂C(═O)NH(CH₂)₂NH₂, HOC(═O)(CH₂)₂NHC(═O)CH₂NH₂, HOC(═O)(CH₂)₂O(CH₂)₃NH₂ and HOC(═O)(CH₂)₂C(═O)NH(CH₂)₂NH₂. Especially preferred linkers include HOC(═O)(CH₂)₅NH₂ and HOC(═O)CH₂C(═O)NH(CH₂)₂NH₂.

These linkers are also suitable for use in other methods of preparing the constrained peptides as described in Schemes 1 and 2.

In the above embodiments, the peptide prepared in the method also comprises a second amino acid residue capable of coupling with the uncoupled amino or carboxylic acid group of the linker to form an amide bond. In preferred embodiments, the second amino acid residue is selected from L-aspartic acid, L-glutamic acid, D-aspartic acid, D-glutamic acid, L-lysine, ornithine or D-lysine. In preferred embodiments, the amino acid prepared in step (i) of the method and the second amino acid residue capable of coupling with the uncoupled amino or carboxylic acid group of the linker are positioned in the peptide in an i(i+7) relationship.

The amino acid may be incorporated in a peptide using solid phase peptide synthesis or solution phase peptide synthesis. In preferred embodiments, solid phase peptide synthesis is used.

In preferred embodiments, the amino acid from step (i) comprises protecting groups for the amino group and carboxylic acid group that do not form part of the side chain of the amino acid, for example, the alpha amino and carboxylic acid groups in an alpha amino acid. Suitable protecting groups include selective protecting groups that may be removed in the presence of other protecting groups. In some embodiments, the alpha carboxylic acid is protected with a protecting group that may be removed without removing the alpha amino protecting group and optionally the protecting group on the uncoupled terminus of the linker. Such protecting groups could be readily ascertained by those skilled in peptide synthesis. One example of a suitable alpha carboxylic acid protecting group is t-butyl. Preferred alpha amino protecting groups are those that can withstand deprotection conditions used to remove any alpha carboxylic acid protection and can be deprotected without removal of the protecting group on the uncoupled terminus of the linker. Such protecting groups could be readily ascertained by those skilled in peptide synthesis. One example of a suitable alpha amino protecting group is Fmoc. This protecting group is particularly suitable for use during solid phase synthetic procedures. The unreacted end of the linker may be protected with any suitable protecting group to prevent unwanted side reactions during peptide synthesis. In some embodiments, this protecting group is able to withstand the conditions used to remove the protecting groups present on the alpha amino group and optionally the alpha carboxylic acid group. When the unreacted terminus of the linker is an amino group, suitable protecting groups include but are not limited to BOC and trityl. When the unreacted terminus of the linker is a carboxylic acid group, suitable protecting groups include but are not limited to t-butyl and optionally substituted phenyl groups.

Amide bond formation between the amino acid in step (i) and the linker or between the deprotected uncoupled end of the linker and the second amino acid residue side chain may be achieved by any means known in the art for amide bond formation in amino acids or peptides. In general, the carboxylic acid group is activated towards nucleophilic attack by an amino nitrogen atom. The carboxylic acid may be activated by formation of an acyl halide, an acyl azide, an acid anhydride or by reaction with a dicarbodiimide reagent by known techniques. In one embodiment of the method of the invention, the amide bond between one or both of the amino acid side chains and one or both termini of the linker is performed using O-benzotriazole-N,N,N′,N′-tetramethyl-uronium hexafluorophosphate (HBTU) and diisopropylethylamine (DIPEA).

The conformationally constrained peptide moiety can be coupled to the cell targeting moiety by any means known in the art suitable for coupling peptides with proteins or other peptides. For example, the N or C terminus of the conformationally constrained peptide, or any amino acid side chain of the conformationally constrained peptide which has a NH₂ or CO₂H group, such as lysine, glutamic acid or aspartic acid, could be coupled to an COOH or NH₂ group on the cell targeting moiety using any general means for coupling carboxylic acids and amines (Jones, 1992). If the cell targeting moiety is an antibody or protein, care must be taken during any deprotection steps required to avoid denaturation of the antibody protein.

In one method, lysine side chains on the antibody may be reacted with a compound containing an activated carboxylic acid ester that is linked via a spacer to a maleimide ring. The resulting antibody, decorated with multiple maleimide rings, will react selectively and irreversibly with thiols, such as cysteine, incorporated into the conformationally constrained peptide. For example, the antibody may be reacted with an N-hydroxy-succinimide (NHS) activated form of maleimide-ACP or sulfosuccinimidyl-4-[N-maleimidomethyl]-cyclohexane-1-carboxylate (sulfo-SMCC) and the resulting antibody may then be reacted with a cysteine containing conformationally constrained peptide, followed by purification on a desalting column to remove reactants. Alternatively, the antibody may be coupled to the peptide by first reacting the antibody with an NHS-pyridyl disulfide, such as 4-succinimidyloxycarbonyl-α-methyl-α-(2-pyridyldithio)toluene (SMPT) or its water soluble variant LC-SMPT; then reacting the antibody with a cysteine-containing peptide to form a disulfide bond. Such disulfide bonds are cleavable in cells.

Conjugates that comprise a conformationally constrained peptide moiety and a cell-targeting moiety can be produced by any suitable technique known to persons of skill in the art. The present invention, therefore, is not dependent on, and not directed to, any one particular technique for conjugating these moieties.

The manner of attachment of a conformationally constrained peptide moiety to a cell-targeting moiety should be such that the biological activity of each moiety is not substantially inhibited or impaired. A linker or spacer may be included between the moieties to spatially separate them. The linker or spacer molecule may be from about 1 to about 100 atoms in length. In some embodiments, the linker or spacer molecule comprises one or more amino acid residues (e.g., from about 1 to about 50 amino acid residues and desirably 1, 2, 3, 4, 5, 6, 7, 8, 9, 10, 15, 20 amino acid residues). Such linkers or spacers may facilitate the proper folding of the moieties.

Suitably, the conformationally constrained peptide moiety is covalently attached to the cell-targeting moiety. Covalent attachment may be achieved by any suitable means known to persons of skill in the art. For example, a chimeric polypeptide may be prepared by linking polypeptides together using crosslinking reagents. Examples of such crosslinking agents include carbodiimides such as, but not limited to, 1-cyclohexyl-3-(2-morpholinyl-(4-ethyl)carbodiimide (CMC), 1-ethyl-3-(3-dimethylaminopropyl)carbodiimide (EDC) and 1-ethyl-3-(4-azonia-4,4-dimethylpentyl)carbodiimide. Exemplary crosslinking agents of this type are selected from the group consisting of 1-cyclohexyl-3-(2-morpholinyl-(4-ethyl)carbodiimide, (1-ethyl-3-(3-dimethylaminopropyl carbodiimide (EDC) and 1-ethyl-3-(4-azonia-4,4-dimethylpentyl)carbodiimide. Examples of other suitable crosslinking agents are cyanogen bromide, glutaraldehyde and succinic anhydride.

In general, any of a number of homobifunctional agents including a homobifunctional aldehyde, a homobifunctional epoxide, a homobifunctional imidoester, a homobifunctional N-hydroxysuccinimide ester, a homobifunctional maleimide, a homobifunctional alkyl halide, a homobifunctional pyridyl disulfide, a homobifunctional aryl halide, a homobifunctional hydrazide, a homobifunctional diazonium derivative and a homobifunctional photoreactive compound may be used. Also included are heterobifunctional compounds, for example, compounds having an amine-reactive and a sulfhydryl-reactive group, compounds with an amine-reactive and a photoreactive group and compounds with a carbonyl-reactive and a sulfhydryl-reactive group.

Homobifunctional reagents are molecules with at least two identical functional groups. The functional groups of the reagent generally react with one of the functional groups on a protein, typically an amino group. Specific examples of such homobifunctional crosslinking reagents include the bifunctional N-hydroxysuccinimide esters dithiobis(succinimidylpropionate), disuccinimidyl suberate, and disuccinimidyl tartrate; the bifunctional imidoesters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butanediol diglycidyl ether, the bifunctional hydrazides adipic acid dihydrazide, carbohydrazide, and succinic acid dihydrazide; the bifunctional diazoniums o-toluidine, diazotized and bis-diazotized benzidine; the bifunctional alkylhalides N,N′-ethylene-bis(iodoacetamide), N,N′-hexamethylene-bis(iodoacetamide), N,N′-undecamethylene-bis(iodoacetamide), as well as benzylhalides and halomustards, such as α,α′-diiodo-p-xylene sulfonic acid and tri(2-chloroethyl)amine, respectively. Methods of using homobifunctional crosslinking reagents are known to practitioners in the art. For instance, the use of glutaraldehyde as a cross-linking agent is described for example by Poznansky et. al., 1984. The use of diimidates as a cross-linking agent is described for example by Wang, et. al., 1977.

Although it is possible to use homobifunctional crosslinking reagents for the purpose of forming a chimeric or conjugate molecule according to the invention, skilled practitioners in the art will appreciate that it is more difficult to attach different proteins in an ordered fashion with these reagents. In this regard, in attempting to link a first protein with a second protein by means of a homobifunctional reagent, one cannot prevent the linking of the first protein to each other and of the second to each other. Accordingly, heterobifunctional crosslinking reagents are preferred because one can control the sequence of reactions, and combine proteins at will. Heterobifunctional reagents thus provide a more sophisticated method for linking two polypeptides. These reagents require one of the molecules to be joined, hereafter called Partner B, to possess a reactive group not found on the other, hereafter called Partner A, or else require that one of the two functional groups be blocked or otherwise greatly reduced in reactivity while the other group is reacted with Partner A. In a typical two-step process for forming heteroconjugates, Partner A is reacted with the heterobifunctional reagent to form a derivatised Partner A molecule. If the unreacted functional group of the crosslinker is blocked, it is then deprotected. After deprotecting, Partner B is coupled to derivatised Partner A to form the conjugate. Primary amino groups on Partner A are reacted with an activated carboxylate or imidate group on the crosslinker in the derivatisation step. A reactive thiol or a blocked and activated thiol at the other end of the crosslinker is reacted with an electrophilic group or with a reactive thiol, respectively, on Partner B. When the crosslinker possesses a reactive thiol, the electrophile on Partner B preferably will be a blocked and activated thiol, a maleimide, or a halomethylene carbonyl (eg. bromoacetyl or iodoacetyl) group. Because biological macromolecules do not naturally contain such electrophiles, they must be added to Partner B by a separate derivatisation reaction. When the crosslinker possesses a blocked and activated thiol, the thiol on Partner B with which it reacts may be native to Partner B.

An example of a heterobifunctional reagent is N-succinimidyl 3-(2-pyridyldithio)propionate (SPDP) (see for example Carlsson et. al., 1978). Other heterobifunctional reagents for linking proteins include for example succinimidyl 4-(N-maleimidomethyl)cyclohexane-1-carboxylate (SMCC) (Yoshitake et. al., 1979), 2-iminothiolane (IT) (Jue et. al., 1978), and S-acetyl mercaptosuccinic anhydride (SAMSA) (Klotz and Heiney, 1962). All three react preferentially with primary amines (e.g., lysine side chains) to form an amide or amidine group which links a thiol to the derivatised molecule via a connecting short spacer arm, one to three carbon atoms long.

Another example of a heterobifunctional reagent is N-succinimidyl 3-(2-pyridyldithio)butyrate (SPDB) (Worrell et. al., 1986), which is identical in structure to SPDP except that it contain a single methyl-group branch alpha to the sulfur atom which is blocked and activated by 2-thiopyridine. SMPT and SMBT described by Thorpe et. al. 1987, contain a phenylmethyl spacer arm between an N-hydroxysuccinimide-activated carboxyl group and the blocked thiol; both the thiol and a single methyl-group branch are attached to the aliphatic carbon of the spacer arm. These heterobifunctional reagents result in less easily cleaved disulfide bonds than do unbranched crosslinkers.

Some other examples of heterobifunctional reagents containing reactive disulfide bonds include sodium S-4-succinimidyloxycarbonyl-α-methylbenzylthiosulfate, 4-succinimidyl-oxycarbony-α-methyl-(2-pyridyldithio)toluene.

Examples of heterobifunctional reagents comprising reactive groups having a double bond that reacts with a thiol group include SMCC mentioned above, succinimidyl m-maleimidobenzoate, succinimidyl 3-(maleimido)propionate, sulfosuccinimidyl 4-(p-maleimidophenyl)butyrate, sulfosuccinimidyl 4-(N-maleimidomethylcyclohexane)-1-carboxylate and maleimidobenzoyl-N-hydroxysuccinimide ester (MBS).

Other heterobifunctional reagents for forming conjugates of two proteins are described for example by Rodwell et. al. in U.S. Pat. No. 4,671,958 and by Moreland et. al. in U.S. Pat. No. 5,241,078.

Crosslinking of the cell-targeting moiety and the conformationally constrained peptide moiety may be accomplished by coupling a carbonyl group to an amine group or to a hydrazide group by reductive amination.

Coupling of the conformationally constrained peptide and the cell targeting moiety rarely interferes with the recognition site of the cell targeting moiety for it's target molecule. The cell targeting moiety's recognition site is often hydrophobic and does not contain suitable functionality for conjugation with the conformationally constrained peptide.

The ability of a conformationally constrained peptide to be a candidate compound capable of inducing apoptosis or cell death in cells can be assessed by using a screening assay for binding of the peptides to a Bcl-2 family protein. A suitable assay is based on the ability of candidate peptides to disrupt, or compete with, the binding of a Bim BH3 peptide comprising the sequence IAQELRRIGDEFN to a Bcl-2 family protein. The BH3 peptide is preferably labelled. Preferably the Bim BH3 peptide has the sequence:
DLRPEIRIAQELRRIGDEFNETYTRR

In a competitive binding assay, the conformationally constrained peptide competes with a labelled peptide for binding to a Bcl-2 family member protein. The protein may be bound to a solid surface to effect separation of bound protein from the unbound labelled peptides. Alternatively, the competitive binding may be conducted in a liquid phase, and a variety of techniques may be used to detect the binding of the labelled peptides to the protein, as known in the art. The amount of bound labelled peptides may be determined to provide information on the affinity of the test compound to the Bcl-2 family protein. The Bcl-2 family protein is preferably selected from Bcl-2 or its homologues, Bcl-x_{L}, Bcl-w, Mcl-1 or A1. For example, the Bcl-2 family protein may be Bcl-2 ΔC22, Bcl-w ΔC29, Bcl-x_{L} ΔC25 or Mcl-1 ΔC23.

Alternatively, when the Bcl-2 homologue is Mcl-1, the Bim BH3 peptide may be replaced by a BakBH₃ peptide sequence, for example:
PSSTMGQVGRQLAIIGDDINRRYDSE
   or a functional fragment thereof that binds with Mcl-1. Preferably the peptide is labelled. Typically the screening assays described above use one or more labelled molecules. The label used in the assay can provide a detectable signal either directly or indirectly. Various labels that can be used include radioactive moieties, fluorescent compounds, chemiluminescent compounds, bioluminescent compounds and specific binding molecules. Specific binding molecules include pairs such as biotin and streptavidin, digoxin and antidigoxin etc. The binding of such labels to the peptides or proteins used in the assay may be achieved by use of standard techniques in the art.

A variety of other reagents may also be included in the reaction mixture of the assay. These include reagents such as salts, proteins, eg albumin, protease inhibitors and antimicrobial agents.

A preferred assay uses an amplified luminescent proximity homogenous assay in which 6-His tagged (Nickel Chelate) or glutathione S-transferase tagged acceptor beads and streptavidin coated donor beads allow a transfer of singlet oxygen from a donor bead to an acceptor bead when the two beads are bought into close proximity by a binding interaction. In the presence of a competing constrained peptide that binds to the protein used in the assay, the donor and acceptor beads do not come into close proximity and the signal is reduced or eliminated.

To determine specific uptake of the antibody-linked peptide into the target cell, a cell line expressing the relevant antigen is used. For example, a human CD19 Fitc linked peptide is tested on human B cell tumor lines such as REH, Raji, NALM1. A T cell line such as Jurkat lacking CD19 is used as a control. For mouse CD19 conjugated peptide, internalization is tested using peripheral blood from mice which contain CD19 positive B cells and CD19 negative T cells, granulocytes and red cells. Multiparameter flow cytometry is used to distinguish between uptake of the conjugated peptide in B cells and not in normal T cells and myeloid cells. In vivo efficacy of the antibody-linked peptide is demonstrated on primary mouse B cell tumor models such as Eμ-myc which are CD19 positive. Peptide/antibody internalization is confirmed by confocal microscopy. Cell killing activity is confirmed by incubating the peptide with cell lines and staining for viable cells using propidium iodide. Confirmation that death was by apoptosis is tested by pre-incubation with a caspase inhibitor such as zVAD-fink.

In another aspect of the invention there is provided a method of regulating the death of a cell, comprising contacting the cell with an effective amount of a conjugate comprising at least one cell targeting moiety and at least one conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, or Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I).

In another aspect of the invention there is provided a method of inducing apoptosis in unwanted or damaged cells comprising contacting said damaged or unwanted cells with an effective amount of a conjugate comprising at least one cell targeting moiety and at least one conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I).

It should be understood that the cell which is treated according to a method of the present invention may be located ex vivo or in vivo. By “ex vivo” is meant that the cell has been removed from the body of a subject wherein the modulation of its activity will be initiated in vitro. For example, the cell may be a cell which is to be used as a model for studying any one or more aspects of the pathogenesis of conditions which are characterised by aberrant cell death signaling. In a preferred embodiment, the subject cell is located in vivo.

In another aspect of the invention there is provided a method of treatment and/or prophylaxis of a pro-survival Bcl-2 family member-mediated disease or condition, in a mammal, comprising administering to said mammal an effective amount of a conjugate comprising at least one cell targeting moiety and a conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I).

In another aspect of the invention there is provided a method of treatment and/or prophylaxis of a disease or condition characterised by the inappropriate persistence or proliferation of unwanted or damaged cells in a mammal, comprising administering to said mammal an effective amount of a conjugate comprising at least one cell targeting moiety and a conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I).

In yet another aspect of the invention there is provided a conjugate comprising at least one cell targeting molecule and at least one conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
   wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I), for use in a method of treatment and/or prophylaxis.

In yet another embodiment of the invention there is provided a use of a conjugate comprising at least one cell targeting moiety and at least one conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
   wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I), for regulating the death of a cell, or for inducing apoptosis in unwanted or damaged cells, or for the treatment and/or prophylaxis of a pro-survival Bcl-2 family member-mediated disease or condition, or for the treatment and/or prophylaxis of a disease or condition characterised by the inappropriate persistence or proliferation of unwanted or damaged cells.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

As used herein, the term “pro-survival Bcl-2 family member-mediated disease or condition” refers to diseases or conditions where unwanted or damaged cells are not removed by normal cellular process, or diseases or conditions in which cells undergo aberrant, unwanted or inappropriate proliferation. Such diseases include those related to inactivation of apoptosis (cell death), including disorders characterised by inappropriate cell proliferation. Disorders characterised by inappropriate cell proliferation include, for example, inflammatory conditions such as inflammation arising from acute tissue injury including, for example, acute lung injury, cancer including lymphomas, such as prostate hyperplasia, genotypic tumours, autoimmune disorders, tissue hypertrophy etc.

Specific antibodies may be used to target specific cells and therefore diseases or conditions that are related to unwanted or damaged cells that are targeted or the proliferation of such cells. For example, antibodies CD19, CD20, CD22 and CD79a are able to target B cells, therefore can be used to deliver the conformationally constrained BH3-only mimic to a B cell to regulate apoptosis in unwanted or damaged B cells. Disorders and conditions that are characterised by unwanted or damaged B cells or the unwanted proliferation of B cells include B cell non-Hodgkins Lymphoma, B cell acute lymphoblastic leukemia (B-ALL) and autoimmune diseases related to B cells such as rheumatoid arthritis, systemic Lupus erythematosis and related arthropathies. Antibodies such as CD2, CD3, CD7 and CD5 are able to target T cells and therefore can be used to deliver the conformationally constrained BH3-only mimic to a T cell to regulate apoptosis in unwanted or damaged T cells. Disorders and conditions that are characterised by unwanted or damaged T cells or the unwanted proliferation of T cells include T cell acute lymphoblastic leukemia (T-ALL), T cell non-Hodgkins Lymphoma and T cell mediated autoimmune diseases such as Graft vs Host disease. Antibodies CD13 and CD33 are able to target myeloid cells and therefore can be used to deliver the conformationally constrained BH3-only mimic to a myeloid cell to regulate apoptosis in unwanted or damaged myeloid cells. Diseases and conditions that are characterised by unwanted or damaged myeloid cells or the unwanted proliferation of myeloid cells include acute myelogenous leukemia (AML), chronic myelogenous leukemia (CML) and chronic myelomonocytic leukemia (CMML). The antibody CD138 is able to target plasma cells therefore can be used to deliver the conformationally constrained BH3-only mimic to plasma cells to regulate apoptosis in unwanted or damaged plasma cells. Diseases and conditions that are characterised by unwanted or damaged plasma cells or the unwanted proliferation of plasma cells include multiple myeloma.

Other cell targeting moieties can also be used to target specific cells. Luteinizing hormone-releasing hormone (LHRH) receptor is expressed in several types of cancer cells, such as ovarian cancer cells, breast cancer cells and prostate cancer cells, but is not expressed in healthy human viceral organs. LHRH can be used as a cell targeting moiety to deliver the conformationally constrained BH3-only mimic to cells expressing LHRH receptor. Disorders or conditions that are able to be treated with a conjugate comprising an LHRH-cell-targeting moiety and a conformationally constrained peptide moiety include ovarian cancer, breast cancer and prostate cancer.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the conjugates of the invention together with the subjection of the mammal to other agents or procedures which are useful in the treatment of diseases and conditions characterised by the inappropriate persistence or proliferation of unwanted or damaged cells. For example, the conjugates of the present invention may be administered in combination with other chemotherapeutic drugs, or with other treatments such as radiotherapy.

Suitable pharmaceutically acceptable salts of the conformationally constrained peptides include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that many of the conjugates, cell targeting moieties or conformationally constrained peptide moieties, of the invention possess asymmetric centres and are therefore capable of existing in more than one stereoisomeric form. The invention thus also relates to conjugates in substantially pure isomeric form at one or more asymmetric centres eg., greater than about 90% ee, such as about 95% or 97% ee or greater than 99% ee, as well as mixtures, including racemic mixtures, thereof. Isomers of the conformationally constrained peptide moieties may be prepared by asymmetric synthesis, for example using chiral intermediates, or by chiral resolution.

The term “prodrug” is used in its broadest sense and encompasses those derivatives that are converted in vivo to the compounds of the invention. Such derivatives would readily occur to those skilled in the art, and include N-α-acyloxy amides, N-(acyloxyalkoxy carbonyl)amine derivatives and α-acyloxyalkyl esters of phenols and alcohols. A prodrug may include modifications to one or more of the functional groups of a conjugate of the invention.

The term “prodrug” also encompasses the use of fusion proteins or peptides comprising cell-permeant proteins or peptides and the conjugates of the invention. Such fusion proteins or peptides allow the translocation of the conjugates of the invention or the conformationally constrained peptide moieties across a cellular membrane and into a cell cytoplasm or nucleus. Examples of such cell-permeant proteins and peptides include the membrane permeable sequences, cationic peptides such as protein transduction domains (PTD), eg: antennapedia (penetratin), tat peptide, R7, R8 and R9, and other drug delivery systems (see Dunican and Doherty, 2001; Shangary and Johnson, 2002; Letai et. al., 2002; Wang et. al., 2000; Schimmer et. al., 2001; Brewis et. al., 2003; Snyder et. al., 2004).

The term “prodrug” also encompasses the combination of lipids with the conjugates of the invention. The presence of lipids may assist in the translocation of the conjugates across a cellular membrane and into a cell cytoplasm or nucleus. Suitable lipids include fatty acids which may be linked to the conjugate by formation of a fatty acid ester. Preferred fatty acids include, but are not limited to, lauric acid, caproic acid, palmitic acid and myristic acid.

The phrase “a derivative which is capable of being converted in vivo” as used in relation to another functional group includes all those functional groups or derivatives which upon administration into a mammal may be converted into the stated functional group. Those skilled in the art may readily determine whether a group may be capable of being converted in vivo to another functional group using routine enzymatic or animal studies.

While it is possible that, for use in therapy, a conjugate of the invention may be administered as a neat chemical, it is preferable to present the active ingredient as a pharmaceutical composition.

The invention thus further provides a pharmaceutical composition comprising a conjugate comprising at least one cell targeting moiety and at least one conformationally constrained peptide moiety, or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′

wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;

Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1; wherein a conformational constraint is provided by a linker which tethers two amino acid residues, Zaa₁ and Zaa₂, in the sequence; and wherein the cell targeting moiety and the conformationally constrained peptide moiety or pharmaceutically acceptable salt or prodrug thereof are coupled through R or R′ or a functionalized amino acid side chain in the amino acid sequence (I), together with one or more pharmaceutically acceptable carriers and optionally, other therapeutic and/or prophylactic ingredients. The carrier(s) must be “acceptable” in the sense of being compatible with the other ingredients of the composition and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The conjugates of the invention, together with a conventional adjuvant, carrier, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to two hundred (200) milligrams, per tablet, are accordingly suitable representative unit dosage forms. The conjugates of the present invention can be administered in a wide variety of oral and parenteral dosage forms. It will be obvious to those skilled in the art that the following dosage forms may comprise, as the active component, either a conjugate of the invention or a pharmaceutically acceptable salt or derivative of the conjugate of the invention.

For preparing pharmaceutical compositions from the conjugates of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired. The powders and tablets preferably contain from five or ten to about seventy percent of the active conjugate. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The conjugates according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis the conjugates according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds according to the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the conjugate in a suitable powder base such as lactose, starch, starch derivatives such as hydroxypropylmethyl cellulose and polyvinylpyrrolidone (PVP).

Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the conjugate formulation will generally have a small particle size for example of the order of 1 to 10 microns or less. Such a particle size may be obtained by means known in the art, for example by micronization.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Liquids or powders for intranasal administration, tablets or capsules for oral administration and liquids for intravenous administration are preferred compositions.

The invention will now be described with reference to the following examples which illustrate some preferred aspects of the present invention. However, it is to be understood that the particularity of the following description of the invention is not to supersede the generality of the preceding description of the invention.

### EXAMPLES

Dynamics Simulations

Molecular dynamics simulations were performed using the GROMACS v. 3.1.1 package of programs [Lindahl, 2001 #1629] with the Gromacs force field (ffgmx2). The simple point charge model for water [Berendsen, 1981 #1620] was used to describe the solvent. Ionisable amino acids were assumed to be in their standard state at neutral pH. Proteins were solvated in a cubic box of water of dimensions of 35³; no pressure coupling was applied. The total charge on the system was made neutral by replacing water molecules with sodium or chloride ions using the GENION procedure. The LINCS algorithm [Hess, 1977 #1624] was used to constrain bond lengths. Protein, water and ions were coupled separately to a thermal bath at 300 K using a Berendsen thermostat [Berendsen, 1984 #1621] applied with a coupling time of 0.1 ps. All simulations were performed using single non-bonded cut-off of 10 Å, applying a neighbour-list update frequency of 10 steps (20 fs). The particle-mesh Ewald method was applied to deal with long-range electrostatics with a grid width of 1.2 Å and a cubic interpolation scheme. All simulations consisted of an initial minimization to avoid close contacts, followed by 1 ps of ‘positional restrained’ molecular dynamics to equilibrate the water molecules (with the protein fixed). Calculations were run for a total simulation time of 50 ns using a time step of 2 fs.

Circular Dichroism

Circular dichroism spectra were obtained using a Jasco Model J-710 spetropolarimeter at 20° C. using the following parameters: path length, 2 mm; step resolution, 0.1 nm; speed, 20 nm/min, accumulation, 4; response, 1 second; bandwidth, 1.0 nm. The peptides were analysed at a concentration of 0.5 mg/mL in 30% aqueous TFE. The alpha-helical content of the peptides were determined by methods described in Yang et al (1986), involving comparisons of spectra with model helical peptides.

Peptide Synthesis

Peptides were prepared by New England Peptides, Inc, (USA) using a Pioneer peptide synthesizer or Proteomics International Pty. Ltd. (ABN 78 096 013 455; Perth, Western Australia) using an Applied Biosystems 433 peptide synthesiser using standard F-moc chemistry (Fields et al., 1991). Amino acid coupling cycles were based on the manufacturers standard protocols. Each peptide was provided with quality assurance data.

The Bim BH3-26 mer peptide used in the assays was prepared by standard solid-phase peptide synthesis techniques using Fmoc chemistry.

Measurement of Competition of Constrained Peptides with Bim26mer

Alphascreen (Amplified Luminsecent Proximity Homogenous Assay) is a bead based technology which measures a biological interaction between molecules. The assay consists of two hydrogel coated beads which, when bought into close proximity by a binding interaction, allow a transfer of singlet oxygen from a donor bead to an acceptor bead.

Upon binding a photosensitiser in the donor bead converts ambient oxygen to a more excited singlet state. This singlet oxygen then diffuses across to react with a chemiluminescer in the acceptor bead. Fluorophores within the same bead are activated, resulting in the emission of light.

Screening of the conformationally constrained peptides was performed using the Hexa-His detection system. Non biotinylated peptides dissolved in DMSO were titrated into the assay which consisted of 6-His tagged Bcl w delta C10 protein (24 nM Final concentration) and Biotinylated Bim BH3-26 peptide, Biotin-DLRPEIRIAQELRRIGDEFNETYTRR (1.5 nM Final concentration). To this reaction mix 6H is tagged (Nickel Chelate) acceptor beads and Streptavidin coated donor beads, both at 10 ug/ml Final concentration, were added.

Assay buffer contained 50 mM Hepes pH 7.4, 10 mM DTT, 100 mM NaCl, 0.05% Tween and 1 mg/ml BSA. Bead dilution buffer contained 50 mM Tris, pH 7.5, 0.01% Tween and 1 mg/ml BSA. The final DMSO concentration in the assay was 1%. Assays were performed in 384 well white Optiplates and analysed on the Perkin Elmer Fusion plate reader (Ex680, Em520-620 nM).

The Alphascreen 6-His detection kit and Optiplates were purchased from Perkin Elmer.

Alternatively, the detection system used was a glutathione S-transferase (GST) detection system and the assay was performed as follows:

Measurement of Competition of Constrained Peptides with Bim26mer Alphascreen (Amplified Luminescent Proximity Homogenous Assay) is a bead based technology which measures a biological interaction between molecules. The assay consists of two hydrogel coated beads which, when bought into close proximity by a binding interaction, allow a transfer of singlet oxygen from a donor bead to an acceptor bead.

Upon binding and excitation with laser light at 680 nm a photosensitiser in the donor bead converts ambient oxygen to its excited singlet state. This singlet oxygen then diffuses across to react with a chemiluminescer in the acceptor bead. Fluorophores within the same bead are activated, resulting in the emission of light at 580-620 nm.

Screening of the conformationally constrained peptides was performed using the AlphaScreen GST (glutathione S-transferase) detection kit detection system. Non biotinylated peptides dissolved in DMSO were titrated into the assay which consisted of GST tagged Bcl w delta C29 protein (0.1 nM Final concentration) and Biotinylated Bim BH3-26 peptide, Biotin-DLRPEIRIAQELRRIGDEFNETYTRR (3.0 nM Final concentration). To this reaction mix anti-GST coated acceptor beads and Streptavidin coated donor beads, both at 10 ug/ml Final concentration, were added and the assay mixture incubated for 4 hours at room temperature before reading.

Assay buffer contained 50 mM Hepes pH 7.4, 10 mM DTT, 100 mM NaCl, 0.05% Tween and 0.1 mg/ml casein. Bead dilution buffer contained 50 mM Tris, pH 7.5, 0.01% Tween and 0.1 mg/ml casein. The final DMSO concentration in the assay was 0.5%. Assays were performed in 384 well white Optiplates and analysed on the PerkinElmer Fusion alpha plate reader (Ex680, Em520-620 nM).

The GST Alphascreen detection kit and Optiplates were purchased from PerkinElmer.

Affinity measurements and solution competition assays (Biacore Assay).

Affinity measurements were performed on a Biacore 3000 biosensor (Biacore) with HBS (10 mM HEPES pH 7.2, 150 mM NaCl, 3.4 mM EDTA, 0.005% Tween-20) as the running buffer. CM5 sensorchips were immobilized with mouse 26-mer wtBimBH3, and 4EBimBH3 mutant peptides using amine-coupling chemistry. To directly assess the binding affinities of pro-survival Bcl-2-like proteins for BimBH3, the proteins were directly injected into the sensorchip at 20 ml/min. After each binding measurement, residual bound protein was desorbed from the chip by injecting 50 mM Sodium Hydroxide or 6 M Guanidium Hydrochloride (pH 7.2), followed by two washes with running buffer. Binding kinetics were derived from sensorgrams, following subtraction of baseline responses, using the BIA evaluation software (version 3, Biacore). The relative affinities of BH3 peptides for pro-survival Bcl-2 proteins were assessed by comparing their abilities to compete for wtBimBH3 peptide binding to Bcl-2-like proteins. The competition binding assays were performed by incubating a fixed sub-saturating amount (10 nM) of pro-survival Bcl-2 protein with varying amounts of competitor BH3 peptide in HBS for at least 2 hr on ice. The mixtures were then injected over a sensorchip containing a channel immobilized with mouse wtBimBH3 and a control one immobilized with mouse 4EBimBH3. The baseline response (from the control channel) was subtracted to obtain the absolute binding response. Taking the response from unbound protein as the maximal response (100%), we calculated the relative residual binding (%) in the presence of increasing amounts of the competitor peptides at a given injection time point (430.5 s). The relative residual responses (f) were plotted against the initial peptide concentrations and fitted to the equation f=100/(1+(c/IC50)m), where c=concentration of the competitor peptide, m=the curvature constant, and IC50=concentration of competitor peptide required to reduce binding by 50%.

Antibody Production

Suitable Antibodies may be prepared by techniques known in the art. See, for example, Galfre et. al., 1977.

Coupling of Antibodies and Conformationally Constrained Peptides.

The antibody is reacted with NHS-activated-maleimide-ACP, sulfosuccinimidyl-4-[N-maleimidomethyl]cyclohexane-1-carboxylate, 4-succinimidyloxycarbonyl-α-methyl-o-(2-pyridyldithio)toluene or LC-SMPT to prepare an antibody decorated with multiple linkers. The antibody is then reacted with a cysteine-containing conformationally constrained peptide.

Cell Based Assay

The efficacy of the conjugates of the present invention can also be determined in cell based killing assays using a variety of cell lines and mouse tumor models. For example, their activity on cell viability can be assessed on a panel of cultured tumorigenic and non-tumorigenic cell lines, as well as primary mouse or human cell populations, e.g. lymphocytes. For these assays, 5,000-20,000 cells are cultured at 37° C. and 10% CO₂ in appropriate growth media, eg: 100 μL Dulbecco's Modified Eagle's medium supplemented with 10% foetal calf serum, asparaginase and 2-mercaptoethanol in the case of pre-B Eμ-Myc mouse tumors in 96 well plates. Cell viability and total cell numbers can be monitored over 1-7 days of incubation with 1 nM-100 μM of the conjugates to identify those that kill at IC50<10 μM. Cell viability is determined by the ability of the cells to exclude propidum iodide (10 μg/mL by immunofluorescence analysis of emission wavelengths of 660-675 nm on a flow cytometer (BD FACScan). Alternatively, a high throughput calorimetric assay such as the Cell Titre 96. AQueous Non-Radioactive Cell Proliferation Assay (Promega) may be used. Cell death by apoptosis is confirmed by pre-incubation of the cells with 50 μM of a caspase inhibitor such as zVAD-fmk. Drug internalisation is confirmed by confocal microscopy of conjugates labelled with a fluorochrome such as Fitc.

The conjugates of the present invention can also be evaluated for the specificity of their targets and mode of action in vivo. For example, if a conjugate comprises a conformationally constrained peptide moiety that binds with high selectivity to Bcl-2, it should not kill cells lacking Bcl-2. Hence, the specificity of action can be confirmed by comparing the activity of the conjugate in wild-type cells with those lacking Bcl-2, derived from Bcl-2-deficient mice.

### Example 1

To investigate synthetically even a fraction of the possible linkers would be prohibitively expensive. Rather, this is a task that lends itself to prior theoretical investigation using molecular dynamics. When an adequate (eg 30 ns) simulation time is used such that several folding and unfolding events are observed, and when solvent is explicitly accounted for, molecular dynamics has been shown to be a useful predictive tool for peptide conformation (Burgi et al 2001).

Molecular dynamics simulations of length 50 nanoseconds were run on the linear Bim-like 12-mer (a) and constrained analogues (c) and (d), a 13-mer (b), and a 16-mer (e) and constrained analogues (f), (g) and (h), using explicit water, in order to see which, if any, type and position of the linker would encourage helix formation. Linkers in (c) and (f) correspond to a 1^{st} position linker as shown in formula (II) above, (d) and (g) to a 2^{nd} position constraint as shown in formula (IV) above, and (h) to a 3 position as shown in formula (VI) above, with the i(i+7) constraint corresponding to residues 94(101):

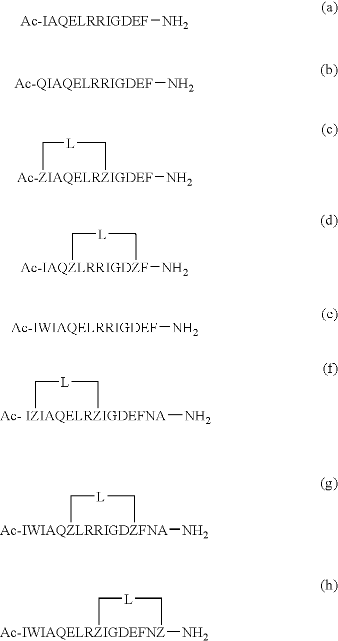

Here, Z indicates the position of the linker that connects two amino glutamic acid residues through their carboxylic acid groups. The linkers investigated were linkers —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—.

Dynamics simulations were run with the 12mer at both the 1^{st} and second positions for linkers —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, otherwise only the second position was investigated.

The dynamics simulations indicated that:

1. The unconstrained 12-mer, (a) Ac-IAQELRRIGDEF-NH₂, was relatively helically unstable.

2. The 12-mer constrained in the 1^{st} position, (c) above, was helically a little more stable, for all linkers looked at, but tended to unravel at the C-terminus after the glycine. An exception was linker —NH(CH₂)₂S(CH₂)₂NH—, which destablized helix formation and seemed even a little worse than the linear (unconstrained) control 12-mer (a).

3. The 12-mer constrained in the 2^{nd} position, (d) above, was generally much more helical than when constrained in the 1^{st} position. In particular, the diaminopentane linker, the diaminoheptane linker, and linkers —NHCH₂C(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH— appeared to be excellent helix-stabilizing linkers. However, the diaminohexane linker, and linkers —NH(CH₂)₂S(CH₂)₂NH—, —NH(CH₂)₂SS(CH₂)₂NH—, —NH(CH₂)₂S(CH₂)₃NH—, —NH(CH₂)₂NHC(═O)(CH₂)₂NH—, —NHCH₂C(═O)NH(CH₂)₃NH—, —NH(CH₂)₃NHC(═O)CH₂NH—, —NH(CH₂)₂C(═O)NH(CH₂)₃NH—, and —NH(CH₂)₃C(═O)NH(CH₂)₂NH— were not as good at stabilizing helix formation.

4. Simulations with the 16-mer (e) generally mirrored these results.

5. The pentane linker in the 3^{rd} position of the 16-mer (h) was a little helix stabilizing, but not as good as when in the 2^{nd} position.

### Example 2

The cyclic peptide Acetyl-IAQ(E1)LRRIGD(E2)F-amide was synthesised using Fmoc chemistry with HTBU activation on an Applied Biosystems Pioneer peptide synthesizer. The resin used during solid phase peptide synthesis was Pal-Peg-PS resin. The base peptide was prepared using orthogonal protection on the glutamic acid residues, (E1=ODMAB, O-4-{N-[1-(4,4-dimethyl-2,6-dioxocyclohexylidene)-3-methylbutyl]amino}benzyl) and (E2=O-2-PhiPR). After synthesis E2 was deprotected selectively while the peptide was still on the resin, and a 1,5-diaminopentane (mono-Fmoc protected) linker was added to the free side chain carboxyl group. Next, the Fmoc was removed, E1 was selectively deprotected and coupled to the diaminopentane linker. The remaining protecting groups and the resin were cleaved using TFA, water, and thiol based scavengers. The peptide was then purified using RP-HPLC on a C18 YMC column. MALDI-TOF DE mass spectral analysis gave M+1: 1555.

### Example 3

The peptide Ac-IAQ-E-LRRIGD-E-F-NH₂ having a 1,6-diaminohexane linker linking the two glutamic acid residues was synthesized and purified as described in Example 2 above but using a 1,6-diaminohexane linker. MALDI-TOF DE mass spectral analysis gave M+1: 1571.

### Example 4

The peptide Ac-E-IAQELR-E-IGDEF-NH₂ having a 1,5-diaminopentane linker linking the two glutamic acid residues was synthesized and purified as described in Example 2 above. MALDI-TOF DE mass spectral analysis gave M+1: 1657.

### Example 5

The preparation of linker precursor NH₂CH₂CC(═O)NHCH₂CH₂NH-Fmoc was synthesized from commercially available compounds Fmoc-NH(CH₂)₂NH₂.HCl (1.9 g 6 mmol) and t-Boc-Gly-Osu (1.6 g, 6 mmol), were dissolved in DMF (15 mL), then treated with N-ethyl-N,N-diisopropylamine (20.1 mL, 12 mmol) and stirred for 2 hours. Water (40 mL) was added to precipitate the product, t-Boc-NH₂CH₂C(═O)NHCH₂CH₂NH-Fmoc, a colourless powder after filtering and air-drying. This was then dissolved in 4M HCl/Ether (15 mL) and stood for 2 hours. The supernatant was decanted and the remaining while granules washed with ether, filtered and dried, giving the product HCl.NH₂CH₂C(═O)NHCH₂CH₂NH-Fmoc in 33% overall yield for the two steps. MS (m/z=340). ¹H NMR (300 MHz, DMSO) δ: 8.51 (broad triplet, 1H, NH); 8.14 (broad singlet, 3H, NH₃); 7.3-7.9 (multiplet, 8H+1H, ArH (Fmoc)+NH); 4.15-4.35 (multiplet, 3H, CH₂CH (Fmoc)); 3.49, (singlet, 2H, CH₂ (gly)); 3.15 (triplet, 2H, CH₂); 3.05 (triplet, 2H, CH₂). Chemical shift (δ) are measured in parts per million (ppm).

### Example 6

The peptide Ac-IAQ-E-LRRIGD-E-F-NH₂ having a —NHCH₂C(═O)NHCH₂CH₂NH— linker linking the two glutamic acid residues was synthesized analogously to Example 2 but using the mono-Fmoc protected linker described in Example 5, except that E1 was selectively deprotected first and reacted with the mono-Fmoc protected linker. The Fmoc was then removed and E2 was deprotected and coupled to the linker.

### Example 7

Four constrained peptides were synthesized as described in Examples 2 to 6, corresponding to the pentane linker in the first position (A), the pentane linker in the second position (B), the hexane linker in the second position (C), and linker —NHCH₂C(═O)NH(CH₂)₂NH— in the second position (D).

Their circular dichroism spectra were measured as a gauge of their helicity in 30% aqueous trifluoroethanol (TFA), and their affinity to Bcl-2 ΔC22, Bcl-w ΔC10 and Bcl-w-ΔC29 measured by means of a competition assay using biotinylated Bim-BH3 peptide. The results are shown below:

| | | IC₅₀ (nM) | IC₅₀ (nM) | IC₅₀ (nM) |
|---|---|---|---|---|
| Peptide | % Helicity | Bcl-2 ΔC22 | Bcl-w ΔC10 | Bcl-w ΔC29 |
| Linear 12mer | 9 | 240,000 | 870 | 4,700 |
| A | 33 | 26,000 | 2,600 | 1,800 |
| B | 28 | 290 | 65 | 150 |
| C | 39 | 2,600 | 230 | 120 |
| D | 16 | 6,900 | 40 | 160 |

The circular dichroism spectra indicated that the constrained peptides were in general more helical—some much more so—than the linear 12-mer. Peptides B and C displayed outstanding increases in affinity for Bcl-2 and Bcl-xL over the unconstrained 12-mer. These sorts of peptides form the basis of the current claim.

### Example 8

The linear 16-mer peptide based on the Bim BH3-only protein, Ac-IWIAQELRRIGDEFNA-NH₂ was prepared using a Pioneer Peptide Synthesizer and purified by HPLC. The constrained peptides were synthesized as described in Examples 2 to 6. The first constrained peptide (E) has a pentane linker tethering the two glutamate residues. The second constrained peptide (F) has a —NHCH₂C(═O)(CH₂)₂NH₂— linker tethering the two glutamic acid residues.

The affinity of linear 16-mer and peptides (E) and (F) for Bcl-w ΔC29 was measured by means of a competition assay using biotinylated BIM-BH3 peptide. The results are shown below.

| | IC50 (nM) | Mass Spectrometry |
|---|---|---|
| Peptide | Bcl-w ΔC29 | MW |
| linear 16-mer | 2.5 | 1972 |
| E | 0.5 | 2037 |
| F | 0.3 | 2054 |

The constrained 16-mer peptides had improved binding affinity with Bcl-w ΔC29.

### Example 9

To ascertain the effect of specific residues in the sequence on binding to Bcl-w ΔC29, substitutions were made in the sequence and IC₅₀ values measured. The peptides used, with the exception of Peptide G, were linear peptides synthesized on a Pioneer peptide synthesiser or Applied Biosystems 433 Peptide Synthesiser using standard F-moc chemistry, Fields et al. (1991). Amino acid coupling cycles were based on the manufacturers standard protocols. Peptide G is a constrained peptide which has a pentane linker between the two glutamic acid residues and was prepared as described in Examples 2 to 6.

| | | Mass | |
|---|---|---|---|
| | | Spectro- | IC₅₀ nM |
| | | metry | Bcl-w |
| Peptide | Sequence | MW | ΔC29 |
| linear 16-mer | Ac-IWIAQELRRIGDEFNA-NH₂ | 1972 | 2.5 |
| G (constrained) | Ac-QAIAQZLRRIGDZFNA-NH₂ | 1940 | 2.4 |
| H (linear) | Ac-IWIAQQLRRIGDQFNA-NH₂ | 1969 | 3.3 |
| I (linear) | Ac-IWAAQELRRIGDEFNA-NH₂ | 1930 | 360 |
| J (linear) | Ac-IWIAQEARRIGDEFNA-NH₂ | 1930 | 3700 |
| K (linear) | Ac-IWIAQELRRAGDEFNA-NH₂ | 1930 | 7.3 |
| L (linear) | Ac-IWIAQELRRIGDEANA-NH₂ | 1896 | 3500 |
| M (linear) | Ac-IWAAQEARRAGDEANA-NH₂ | 1836 | 64,000 |
| N (linear) | Ac-IFIAQELRRIGDEFNA-NH₂ | 1933 | 11 |
| O (linear) | Ac-AWIAQELRRJGDEFNA-NH₂ | 1930 | 22 |
| P (linear) | Ac-IAIAQELRRIGDEFNA-NH₂ | 1857 | 42 |
| Q (linear) | Ac-IRIAQELRRIGDEFNA-NH₂ | 1942 | 17 |
| R (linear) | Ac-IWIAQELRRIGDEFAN-NH₂ | 1972 | 12 |
| S (linear) | Ac-IWIAQELRRIGDEFAA-NH₂ | 1929 | 3.3 |
| T (linear) | Ac-IWIAQELCitCitIGDEFNA-NH₂ | 1975 | 20 |
| U (linear) | Ac-IWIAQELRRIGDEFNN-NH₂ | 2015 | 5.8 |

Replacement of the first two residues in the constrained peptide (G) with the helix stabilizing QA residues led to a reduction in binding of the constrained peptide (E:0.5 nM, G:2.4 nM), indicating that one or both of the I and W residues interacts favourably with the Bcl-w protein.

The importance of the first two residues I and W can also be seen in the linear peptides. When W→F (peptide N), I→A (peptide O), W→A (peptide P) and W→R (peptide Q) substitutions are made, there is also a drop in binding compared to the linear 16-mer.

To confirm that it was the constraint that provided increased binding activity and not just the loss of two negative charges in the sequence, the two glutamate residues were amidated to provide glutamine residues (peptide I). This resulted in a slight decrease in binding affinity, not an increase.

To show the importance of the hydrophobic residues, each Haa was substituted with alanine. Peptide I (I→A) showed a 100-fold decrease in binding affinity, Peptide J (L→A) showed about 1000-fold decrease in affinity, Peptide K (I→A) showed a 3-fold decrease in affinity and Peptide L (F→A) showed a 1,000-fold decrease in affinity. When all 4 Haa were substituted by alanine there was a 25,000-fold decrease in binding affinity.

Peptide S, Peptide T and Peptide U are substitutions at the last two residues in the sequence. Peptide S (NA→AA) showed only slight, if any, loss of binding affinity, while Peptide U (NA→NN) showed about a two-fold loss. However, when both residues were substituted (by reversal, NA→AN), these losses were more than additive and there is a 4-5-fold decrease in affinity.

### Example 10

Two further peptides related to Puma and Bmf BH3-only proteins were synthesized on a Pioneer peptide synthesizer and their binding affinity for Bcl-2 ΔC26 assessed.

| | | Mass | IC₅₀ nM |
|---|---|---|---|
| | | Spectrometry | Bcl-w |
| Peptide | Sequence | MW | ΔC29 |
| Puma | Ac-REIGAQLRRMADDLNA-NH₂ | 1870 | 52 |
| Bmf | Ac-VQIARKLQAIADQFHR-NH₂ | 1935 | 0.25 |

### Example 11

Bcl-w has been used in Examples 8 to 10 because it is a robust protein to use. However as shown below, when tested for affinity to Bcl-2 ΔC22, Bcl-w ΔC10 and Bcl-w ΔC29 using the Biacore assay and Bcl-w ΔC29 using the Alpha screen assay with GST detection, the Bim-26mer shows similar potency with respect to Bcl-w and Bcl-2. In line with the results shown in example 7, constrained peptides will also potently inhibit the binding of Bim26mer to Bcl-2 and more so than their linear counterparts.

| | | IC₅₀ nM | IC₅₀ nM | IC₅₀ nM | IC₅₀ nM |
|---|---|---|---|---|---|
| | | Bcl-w ΔC29 | Bcl-w ΔC22 | Bcl-w ΔC10 | Bcl-w ΔC29 |
| Peptide | Sequence | Biacore | Biacore | Biacore | Alpha Screen |
| hsBimL/Bod | DMRPEIWIAQELRR | 4.3 | 2.6 | 6 | 0.1 |
| (81-106) | IGDEFNAYYARR | | | | |

### Example 12

A retro inverso peptide having the sequence

Ac-a-n-f-e-d-g-i-r-r-1-e-q-a-i-w-i-NH₂

(Small letters refer to D-amino acids), was synthesised on an Applied Biosystems 433 Peptide Synthesiser using standard F-moc chemistry, Fields et al. (1991). Amino acid coupling cycles were based on manufacturers standard protocols. The peptide was purified by HPLC and molecular weight by mass spectrometry was 1971.

### Example 13

An alternative synthesis of the constrained peptide Ac-IAQZ₁LRRIGDZ₂F-NH₂ in which Z₁ and Z₂ are glutamic acid residues linked through their side chain carboxylic acid groups by a diaminopentane linker was performed, in which the linker was reacted with the glutamic acid before incorporation into the peptide.
FmocGlu (MonoBoc-Diaminoalkyl)-OH Derivative

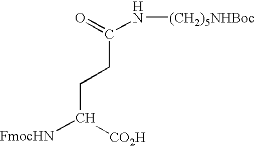

On a 2 mMol scale, Fmoc-Glu-OtBu was coupled through its side chain to NH₂(CH₂)₅NHBoc by standard HBTU/DIPEA coupling in DMF. After a standard organic/aqueous workup with ethyl acetate, the resulting organic layer was concentrated and the residue containing Fmoc-Gln-[(CH₂)₅NHBoc]-OtBu was treated with 50% trifluoro acetic acid (TFA) in dichloromethane (DCM) for an hour. The solution was then concentrated and the residue containing Fmoc-Gln-[(CH₂)₅NH₂.TFA]-OH was dissolved in methanol and filtered through celite. After concentration of the filtrate, the residue was treated with Boc₂O (5 mmol) and DIPEA (10 mmol) in 50% aqueous acetone for 3 hours. After acidification with 10% citric acid, the product was extracted into ethyl acetate and washed with water. The separated organic layer was dried and evaporated to give a gum which was purified through a plug of silia with 10% MeOH/DCM to give Fmoc-Gln-[(CH₂)₅NHBoc]-OH (800 mg) as a gum which became a glass upon exposure to high vacuum. Analysis by positive electrospray mass spectrometry provided a molecular ion of 554, calculated MW 553.

Peptide Synthesis

The peptide IAQZ₁LRRIGDZ₂F, where Z₁ is the above Boc-protected amino pentylglutamine residue and Z₂ is glutamic acid was synthesized using solid phase synthesis on Rink resin using Fmoc protected amino acids and the following protected amino acids Fmoc-Gln-[(CH₂)₅NHBoc]-OH (Z₁), Fmoc-Gln(2-PhiPr)-OH (Z₂), Fmoc-Asp(tBu)-OH, Fmoc-Arg(Pbf)-OH and Fmoc-Gln(trt)-OH. Couplings were performed with standard HBTU/DIPEA coupling conditions. The Fmoc protecting group in each cycle was removed by treatment with 0.2M HOBt/25% piperidine/DMF for 1 minute. After completion of the peptide, the N-terminus was acetylated with acetic anhydride by standard methods.

The resin/peptide Ac-IAQQ[(CH₂)₅NHBoc]LRRIGDE[2-PhiPr]F-Rink was treated with 2% TFA/DCM to deprotect the Z₁ and Z₂ residue side chains and create free amine and carboxylic acid groups. Standard HBTU/DIPEA coupling conditions were employed to complete the linkage between Z₁ and Z₂. The constrained peptide was deprotected and cleaved from the resin using standard deprotection and cleavage conditions to provide an amide protected C-terminus on the peptide.

The constrained peptide was purified by reversed-phase HPLC on a C18 column (Alltech Absorbosphere HS C18 5 μM, 150×3.2 mm) in 0.1% TFA buffers with an acetonitrile gradient (0.0-75% over 25 minutes). The peptide was monitored at 214 nm and was judged to be 95% pure.

The identity of the peptide was confirmed as 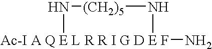
by electrospray mass spectrometry (Micromass Platform 2, sample introduction in 50% acetonitrile/water with a flow of 20 μL/min. The peptide exhibited a doubly charged ion at m/e 777.8 and a triply charged ion at 519.0. This data was transformed to give a MW of 1554.0 (calculated 1553.8).

### Example 14

Using solution competition assays and Alphascreen GST-detection as described below, the constrained peptide of Example 2 (peptide A) was assessed for competition binding to Bcl-2 homologues, Bcl-w ΔC29, Bcl-xL ΔC25 and Mcl-1 ΔC23.

All the assays were performed using 384-well white plates in a total volume of 20 μL. The assay buffer is 50 mM HEPES, 10 mM DTT, 100 mM NaCl, 0.05% Tween 20, 0.1 mg/mL casein, pH 7.4. The bead buffer is 50 mM Tris, 1% Tween 20, 0.1 mg/mL casein, pH 7.5.

For the GST-Bcl-wΔC29 protein assay, protein (0.10 nM), acceptor beads (10 μg/mL), 50% assay buffer and 50% bead buffer were incubated together for 30 minutes. At the same time, biotinylated BimBH3 peptide (Biotin-DLRPEIRIAQELRRIGDEFNETYTRR-OH) (3 nM), donor beads (10 μg/mL), 50% assay buffer and 50% bead buffer were incubated to get for 30 minutes. For the competition binding assay, protein acceptor bead solution (10 μL) and the candidate compound, A, L1 or L2, were added into each well and incubated for 30 minutes, then biotinylated BimBH3 peptide donor bead solution (10 μL) was added into each well. The total DMSO concentration in each well was then adjusted to 0.5% to 2%. Plates were covered with aluminium foil and incubated at room temperature for 4 hours before reading in a Packard Fusion™ reader with excitation at 680 nm and emission at 520-620 nm. Owing to light sensitivity, all assays were carried out under subdued lighting.

For the Mcl-1 assay, the same protocol was adopted using GST-Mcl-1 protein (0.40 nM) and biotinylated BakBH3 peptide (Biotin-PSSTMGQVGRQLAIIGDDINRRYDSE-OH) (4 nM).

For the Bcl-x_{L}ΔC24 assay, the same protocol was adopted using GST-Bcl-x_{L} protein (0.6 nM) and biotinylated BimBH3 peptide (5 nM).

The assays were performed with the linear peptide controls, L1 and L2 and constrained peptide A as candidate compounds. The results are shown below.

| | | Alphascreen IC₅₀ (nM) | | | | | |
|---|---|---|---|---|---|---|---|
| | | Bcl-w | | Bcl-xL | | Mcl-1 | |
| Peptide | Sequence | ΔC29 | | ΔC25 | | ΔC23 | |
| A | Ac-IAQZLRRIGDZF-NH₂ | 40 | 34 | 57 | 54 | 3 | 3 |
| L1 | Ac-IAQELRRIGDEF-NH₂ | 820 | 800 | 7000 | 6300 | 400 | 360 |
| L2 | Ac-IAQQLRRIGDQF-NH₂ | 120 | 120 | 960 | 890 | 650 | 660 |

where Z represents two glutamate residues linked via their side chains with a 1,5-diaminopentane linker.

In separate assay experiments, surface plasmon resonance (SPR) experiments using a Biacore S51 Biosensor were used as proof of direct binding of peptide A to Bcl-w ΔC29, Bcl-xL ΔC25 and Mcl-1 ΔC23. This technique also has the advantage of yielding dissociation constants, which are shown below.

| | Bcl-xL ΔC25 | Mcl-1 ΔC23 |
|---|---|---|
| Peptide | K_{D} (nM) | K_{D} (nM) |
| A | 10 | 10 |
| L1 | 5200 | 1200 |
| L2 | 370 | 920 |

From both sets of experiments, it is clear that constrained peptide A is vastly more potent than its linear counterparts.

### Example 15

Hybridoma line 1 D3 (anti-murine CD19) was grown in hybridoma free medium (Gibco, Invitrogen, USA) containing 1% foetal calf serum (Trace, Australia). Monoclonal antibody (MAb) was purified from culture supernatant using protein G-Sepharose (Amersham—Pharmacia, Sweden) by affinity chromatography according to the manufacturer's instructions. Eluted MAb at 1.35 mg/mL was dialysed against PBS and sterile filtered.

The MAb was reacted via its free lysine side chains with N-hydroxy-succinamide (NHS)-Acp-Maleimide (Sigma 63177) or NHS-pyridyl disulfide (Pierce SMPT 21558 or LC-SMPT 21569). The resulting maleimide tagged MAb was reacted with the thiol group of the cysteine in

Ac-C-Acp-DMRPEIWIAQELRRIGDEFNAY-IARR-NH₂

to give a peptide-MAb conjugate which precipitated upon addition of the peptide to the MAb. The conjugate was analysed by SDS-Page which showed no MAb in the supernatant, the pellet showed MAb of higher molecular weight than control MAb.

### Example 16

150 μL of pre-B tumor cells from E-mu myc transgenic mice in Dulbecco's Modified Eagle's Medium containing 10% Fetal calf serum, 2-mercaptoethanol and asparagine (FMA media) at 4×10⁵/mL concentration in 96 well plates were incubated with 0.02, 0.03, 0.06, 0.13, 0.25 and 0.5 μM CD19 antibody alone, a conjugate of CD19 antibody and linear Bim BH3 peptide having the sequence Ac-C-Acp-DMRPEIWIAQELRRIGDEFNAYYARR-NH₂ prepared in Example 15, or Etoposide. After 24 hours incubation at 37° C. in 5% CO₂, the cells were washed with PBS and 50 μL of 100 μg/mL solution of propidium iodide was added to each well. The cells were analyzed by flow cytometry (BD Facscan) and the viable cells denoted as the percentage of cells excluding propidium iodide. The results are shown below.

% viable cells at 24 hours

| | concentration (μM) | | | | | | | |
|---|---|---|---|---|---|---|---|---|
| | 0 | 0.015 | 0.0312 | 0.0625 | 0.125 | 0.25 | 0.5 | 1.0 |
| Etoposide | 85 | 80 | 50 | 21 | 0 | 0 | 0 | 0 |
| CD19 Ab | 83 | 10 | 6 | 0 | 0 | 0 | 0 | 0 |
| CD19 Ab/Bim | 84 | 6 | 3 | 0 | 0 | 0 | 0 | 0 |
| BH₃ peptide | | | | | | | | |

### Example 17

Animal Models:

To assess the anti-tumour efficacy of the conjugates of the present invention in vivo, the BH3 mimetic conjugates can be given alone (intra-venously; iv or intra-peritoneally; ip) or in combination with sub-optimal doses of clinically relevant chemotherapy (e.g. 25-100 mg/kg cyclophosphamide intra-peritoneally). Mice injected intra-peritoneally with 10⁶ Bcl-2-overexpressing mouse lymphoma cells (Strasser 1996; Adams 1999) develop an aggressive immature lymphoma that is rapidly fatal within 4 weeks if untreated, but are partially responsive to cyclophosphamide. The lymphoma/leukaemia can readily be monitored by performing peripheral blood counts in the animals using a Coulter counter or by weighing the lymphoid organs (lymph nodes, spleen) when the animals are sacrificed. Another model is implantation of a cell line such as that derived from human follicular lymphoma (DoHH2) into immunocompromised SCID mice (Lapidot 1997). Because the conjugates of the invention are contemplated to be efficacious in combination therapy, their in vivo activity can be evaluated alone or in combination with conventional chemotherapeutic agents (e.g. cyclophosphamide, doxorubucin, epipodophylotoxin (etoposide; VP-16)). Cohorts of 18-20 mice per treatment arm will be studied to enable a 25% difference in efficacy with a power of 0.8 at a significance level of 0.05 to be determined. These in vivo tests in mice will also generate preliminary pharmacokinetic, pharmacodynamic and toxicology data.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Adams G P, McCartney J E, Tai M S, Oppermann H, Huston J S, Stafford W F 3^{rd}, Bookman M A, Fand I, Houston L L, Weiner L M, Highly Specific in vivo tumor targeting by monovalent and divalent forms of 741F8 anti-c-erbB-Z single-chain Fr. Cancer Res., 53: 4026-4034, 1993.
Adams J M and Cory S, Oncogene cooperation in leukemogenesis. Cancer Surveys 15:119-141, 1992.
Adams J M and Cory S, The Bcl-2 protein family: arbiters of cell survival. Science 281: 1322-1326, 1998.
Adams, J M, Harris A W, Strasser A, Ogilvy S, and Cory S, Transgenic models of lymphoid neoplasia and development of a pan-hematopoietic vector. Oncogene 18, 5268, 1999.
Adams J M and Cory S, Life-or-death decisions by the Bcl-2 protein family. Trends in Biochemical Sciences 26: 61-66, 2001.
Antonsson B, Montessuit S, Sanchez B and Martinou J C, Bax is present as a high molecular weight oligomer/complex in the mitochondrial membrane of apoptotic cells. Journal of Biological Chemistry 276(15): 11615-11623, 2001
Ashkenazi A and Dixit V M, Death receptors: signaling and modulation. Science 281(5381): 1305-1308, 1998.
Ausubel et al., Current Protocols in Molecular Biology, John Wiley & Sons Inc., 1994-1998.
Baell J B and Huang D C S, Prospects for Targeting the Bcl-2 family proteins to develop novel cytotoxic drugs. Biochem. Pharmacol., 64: 851-863, 2002.
Bouillet P, Metcalf D, Huang D C S, Tarlinton D M, Kay T W H, Kontgen F, Adams J M and Strasser A, Proapoptotic Bcl-2 relative Bim required for certain apoptotic responses, leukocyte homeostasis, and to preclude autoimmunity. Science 286: 1735-1738, 1999.
Bouillet P, Cory S, Zhang L-C Strasser A and Adams J M, Degenerative disorders caused by Bcl-2 deficiency are prevented by loss of its BH3-only antagonist Bim. Developmental Cell 1(5): 645-653, 2001.
Brady L and Dodson G, Reflections on a peptide, Nature, 368 (6473), 692-693, 1994.
Brewis N D, Phelan A, Normand N, Choolun E, O'Hare P, Particle assembly incorporating a VP22-BH3 fusion protein facilitating intracellular delivery, regulated release and apoptosis. Mol. Ther. 7 (2): 262-70, 2003.
Brown J M and Wouters B G, Apoptosis, p53 and tumor cell sensitivity to anticancer agents. Cancer Research 59 (7): 1391-1399, 1999.
Burgi R, Daura X, Mark A, Bellanda M, Mammi S, Peggion E, van Gunsteren W, Folding study of an Aib-rich peptide in DMSO by molecular dynamics simulations, Journal of Peptide Research, 57, 107-118, 2001.
Carlsson J, Drevin H, Axen R, Protein thiolation and reversible protein-protein conjugation, N-succinimidyl 3-(2-pyridyldithio)propionate, a new heterobifunctional reagent. Biochem. J, 173: 723-37, 1978.
Chen F, Hersh B M, Conradt B, Zhou Z, Riemer D, Gruenbaum Y and Horvitz H R, Translocation of C. elegans CED-4 to nuclear membranes during programmed cell death. Science 287(5457): 1485-1489, 2000.
Cheng E H, Wei M C, Weiler S, Flavell R A, Mak T W, Lindsten T and Korsmeyer S J, Bcl-2, Bcl-x_{L} sequester BH3 domain-only molecules preventing BAX and BAK-mediated mitochondrial apoptosis. Molecular Cell 8(3): 705-711, 2001.
Chinnaiyan A M, O'Rourke K, Lane B R and Dixit V M, Interaction of CED-4 with CED-3 and CED-9: a molecular framework for cell death. Science 275(5303): 1122-1126, 1997.
Cleary M L, Smith S D and Sklar J, Cloning and structural analysis of cDNAs for Bcl-2 and a hybrid Bcl-2/immunoglobulin transcript resulting from the t(14;18) translocation. Cell 47(1): 19-28, 1986.
Coligan et. al., Current Protocols in Immunology, John Wiley & Sons, Inc., 1991.
Conradt B and Horvitz H R, The C. elegans protein EGL-1 is required for programmed cell death and interacts with the Bcl-2-like protein CED-9. Cell 93(4): 519-529, 1998.
Conus S, Rosse T and Borner C, Failure of Bcl-2 family members to interact with Apaf-1 in normal and apoptotic cells. Cell Death and Differentiation, 7(10): 947-954, 2000.
Cory S and Adams J M, The Bcl-2 family: Regulators of the cellular life-or-death switch; Nature Reviews Cancer, 2, 647-656, 2002a.
Cumber A J, Ward E S, Winter G, Parnell G D, Wawrzynczak E J, Comparative stabilities in vitro and in vivo of a recombinant mouse antibody FrCys fragment and a BisFrCys conjugate. J. Immunol., 149: 120-126, 1992.
Davies J, Riechmann L, “Camelising” human antibody fragments: NMR studies on VH domains. FEBS Lett., 339: 285-290, 1994.
Dearden C, Monoclonal antibody therapy of haematological malignancies. Biodrug, 1b: 283-301, 2002.
Desagher S, Osen-Sand A, Nichols A, Eskes R, Montessuit S, Lauper S, Maundrell K, Antonsson B and Martinou J-C, Bid-induced conformational change of Bax is responsible for mitochondrial cytochrome c release during apoptosis. Journal of Cell Biology 144(5): 891-901, 1999.
Dharap S S and Minko T, Targeted proapoptotic LHRH-BH3 peptide. Pharm. Res., (6): 889-896, 2003.
Du C, Fang M, Li Y, Li L and Wang X, Smac, a mitochondrial protein that promotes cytochrome c-dependent caspase activation by eliminating IAP inhibition. Cell 102(1): 33-42, 2000.
Dunican D J and Doherty P, Designing Cell-permeant phosphopeptides to modulate intracellular signalling pathways; Biopolymers (Peptide Science), 60, 45-60, 2001.
Eskes R, Desagher S, Antonsson B and Martinou J C, Bid induces the oligomerisation and insertion of Bax into the outer mitochondrial membrane. Molecular and Cellular Biology 2(3): 929-935, 2000.
Fields C G, Lloyd D H, Macdonald R L, Otteson K M, Noble R L, HBTU activation for automated Fmoc solid-phase peptide synthesis, Peptide Research, 4(2), 95-101, 1991.
Fisher D E, Apoptosis in cancer therapy: crossing the threshold. Cell 78: 539-542, 1994.
Galfre G, Howe S C, Milstein C, Butcher G W, Howard J C, Antibodies to major histocompatibility antigens produced by hybrid cell lines. Nature, 266: 550-552, 1977.
Glockshuber R, Malia Mm Pfitzinger I, Plückthun A, A comparison of strategies to stabilise immunoglobulin Fv-fragments. Biochem., 29: 1363-1367, 1990.
Goulet A C, Goldmacher V S, Lambert J M, Baron C, Roy D D, Kouassi E, Conjugation of blocked ricin to an anti-CD19 monoclonal antibody increases antibody-induced cell calcium mobilization an CD19 internalization. Blood, 90 (6): 2364-75, 1997.
Green T W and Wutz P, Protective Groups in Organic Synthesis, John Wiley & Son, 3^{rd} Ed., 1999.
Hamers-Casterman C, Atarhouch T, Muyldermans S, Robinson G, Hamers C, Songa E B, Bendahman N, Hamers R, Naturally occurring antibodies devoid of light chains. Nature, 363: 446-448, 1993.
Han J, Flemington C, Houghton A B, Gu Z, Zambetti G P, Lutz R J, Zhu L and Chittenden T, Expression of bbc2, a pro-apoptotic BH3-only gene, is regulated by diverse cell death and survival signals. Proceedings of the National Academy of Sciences of the USA 98(20): 11318-11323, 2001.
Haraguchi M, Torii S, Matsuzawa S, Xie Z, Kitada S, Krajewski S, Yoshida H, Mak T W and Reed J C, Apoptotic protease activating factor 1 (Apaf-1)-independent cell death suppression by Bcl-2. Journal of Experimental Medicine 191(10): 1709-1720, 2000.
Hausmann G, O'Rielly L A, van Driel R, Beaumont J G, Strasser A, Adams J M and Huang D C S, Pro-apoptotic apoptosis protease-activating factor 1 (Apaf-1) has a cytoplasmic localization distinct from Bcl-2 or Bcl-x(L), J. Cell. Biol., 149(3): 623-34, 2000.
Huang D C S and Strasser A, BH3-only proteins—essential initiators of apoptotic cell death. Cell 103: 839-842, 2000.
Jameson B A, McDonnell J M, Marini J C, Korngold R, A rationally designed CD4 analogue inhibits experimental allergic encephalomyelitis, Nature, 368(6473), 744-746, 1994.
Jones J, Amino acid and peptide synthesis, Oxford Chemistry Primers, Oxford University Press, 1992.
Ju R, Lambert J M, Pierce L R, Traut R R, Addition of sulfhydryl groups to Escherichia coli ribosomes by protein modification with 2-iminothiolane (methyl 4-mercaptobutyrimidate). Biochemistry, 17: 5399-5406, 1978.
Jürgensmeier J M, Xie Z, Deveraux Q, Ellerby L, Bredesen D and Reed J C, Bax directly induces release of cytochrome c from isolated mitochondria. Proceedings of the National Academy of Sciences of the United States of America 95(9): 4997-5002, 1998.
Kamada S, Shimono A, Shinto Y, Tsujimura T, Takahashi T, Noda T, Kitamura Y, Kondoh H and Tsujimoto Y, Bcl-2 deficiency in mice leads to pleiotropic abnormalities: accelerated lymphoid cell death in thymus and spleen, polycystic kidney, hair hypopigmentation, and distorted small intestine. Cancer Research 55: 354-359, 1995.
Klotz I M and Heiney R E, Introduction of sulfhydryl groups into proteins using acetylmercaptosuccinic anhydride. Arch. Biochem. Biophys., 96: 605-612, 1962.
Kluck R M, Bossy-Wetzel E, Green D R and Newmeyer D D, The release of cytochrome c from mitochondria—a primary site for Bcl-2 regulation of apoptosis. Science 275(5303): 1132-1136, 1997.
Köhler G and Milstein C, Continuous cultures of fused cells secreting antibody of predefined specificity. Nature, 256: 495-497, 1975.
Korsmeyer S J, Wei M C, Saito M, Weiler S, Oh K J and Schlesinger P H, Pro-apoptotic cascade activates BID, which oligomerizes BAK or BAX into pores that result in the release of cytochrome c. Cell Death and Differentiation 7(12): 1166-1173, 2000.
Kostelny S A, Cole M S, Tso J Y, Formation of a bispecific antibody by the use of leucine zippers. J. Immunol., 148: 1547-1553, 1992.
Krebber A, Bomhauser S, Burmester J, Honnerger A, Wiluda J, Bosshard H R, Plückthun A, Reliable cloning of functional antibody variable domains from hybridomas and spleen cell repertoires employing a reengineered phage display system. J. Immunol. Methods, 201(1): 35-55, 1997.
Ku J, Schultz P G, Alternate Protein Frameworks for Molecular Recognition. Proc. Natl. Acad. Sci. USA, 92: 6552-6556, 1995.
Lane D P, p53, guardian of the genome. Nature 358: 15-16, 1992.
Lapidot T, Fajerman Y, and Kollet O, Immune-deficient SCID and NOD/SCID mice models as functional assays for studying normal and malignant human hematopoiesis. Journal of Molecular Medicine 75, 664, 1997.
Letai A, Bassik M C, Walensky L D, Sorcinelli M D, Weiler S, Korsmeyer S J, Distinct BH3 domains either sensitize or activate mitochondrial apoptosis, serving as prototype cancer therapeutics. Cancer Cell, 2 (3): 183-92, 2002.
Li P, Nijhawan D, Budihardjo I, Srinivasula S M, Ahmad M, Alnermri E S and Wang X, Cytochrome c and dATP-dependent formation of Apaf-1(Caspase-9 complex initiates an apoptic protease cascade. Cell 91: 479-489, 1997.
Lowe S W, Schmitt E M, Smith S W, Osborne B A and Jacks T, p53 is required for radiation-induced apoptosis in mouse thymocytes. Nature 362: 847-849, 1993.
Ludwig D L, Pereira D S, Zhu Z, Hicklin D J, Bohlen P, Monoclonal antibody therapeutics and apoptosis. Oncogene, 22 (56): 9097-9106, 2003.
Maison W, Arce E, Reynold P, Kennedy R J, Kemp D S, Optimal N-Caps for N-Terminal Helical Templates: Effects of changes in H-Bonding Efficiency and Charge. J. Am. Chem. Soc., 123: 10245-10254, 2001 and references therein.
Marks A J, Cooper M, Orchard K, Folarin N I, Ganeshaguru K, Hoffbrand A V, Mehta A B, Wickremasinghe R G, Targeted therapy with an amphipathic peptide results in selective apoptotic cell death in CD19 positive chronic lymphocytic leukemia cells. Abstract, Blood, (11): 247 Part 1, Nov. 16, 2003.
Marsden V S, O'Connor L, O'Reilly L A, Silke J, Metcalf D, Ekert P G, Huang D C, Cecconi F, Kuida K, Tomaselli K J, Roy S, Nicholson D W, Vaux D L, Bouillet P, Adams J M and Strasser A, Apoptosis initiated by Bcl-2-regulated caspase activation independently of the cytochrome c/Apaf-1/caspase-9 apoptosome; Nature, 419(6907) 634-7, 2002.
Mikhailov V, Mikhailova M, Pulkrabek D J, Dong Z, Venkatachalam M A and Saikumar P, Bcl-2, prevents bax oligomerization in the mitochondrial outer membrane. Journal of Biological Chemistry 20: 20, 2001.
Moriishi K, Huang D C S, Cory S and Adams J M, Bcl-2 family members do not inhibit apoptosis by binding the caspase-activator Apaf-1. Proceedings of the National Academy of Sciences of the United States of America 96: 9683-9688, 1999.
Motoyama N, Wang F P, Roth K A, Sawa H, Nakayama K, Nakayama K, Negishi I, Senju S, Zhang Q, Fujii S and Loh D Y, Massive cell death of immature hematopoietic cells and neurons in Bcl-x deficient mice. Science 267: 1506-1510, 1995.
Motoyama N, Kimura T, Takahashi T, Watanabe T and Nakano T, Bcl-x prevents apoptotic cell death of both primitive and definitive erythrocytes at the end of maturation. Journal of Experimental Medicine 189(11): 1691-1698, 1999.
Nakano K and Wousden K H, PUMA, a novel proapoptotic gene, is induced by p53. Molecular Cell 7:683-694, 2001.
Nakayama K, Nakayama K−1, Negishi I, Kuida K, Sawa H and Loh D Y, Targeted disruption of Bcl-2ab in mice: occurrence of gray hair, polycystic kidney disease, and lymphocytopenia. Proceedings of the National Academy of Sciences of the United States of America 91: 3700-3704, 1994.
Oda E, Ohki R, Murasawa H, Nemoto J, Shibue T, Yamashita T, Tokino T, Taniguchi T and Tanaka N, Noxa, a BH3-only member of the Bcl-2 family and candidate mediator of p53-induced apoptosis. Science 288(5468): 1053-1058, 2000.
Pack P, Plückthun A, Miniantibodies: use of amphipathic helices to produce functional, flexibly linked dimeric FV fragments with high avidity in Escherichia coli. Biochem. 31: 1579-1584, 1992.
Parrish J, Metters H, Chen L and Xue D, Demonstration of the in vivo interaction of key cell death regulators by structure-based design of second-site suppressors. Proceedings of the National Academy of Sciences of the USA 97(22): 11916-11921, 2000.
Petros A M, Nettesheim D G, Wang Y, Olejniczak E T, Meadows R P, Mack J, Swift K, Matayoshi E D, Zhang H, Thompson C B and Fesik S W, Rationale for Bcl-x_{L}/Bad peptide complex formation from structure, mutagenesis and biophysical studies. Protein Science 9: 2528-2534, 2000.
Plückthun et. al., Antibody Engineering: A practical approach. 203-252, 1996.
Poznansky M J, Singh R, Singh B, Fantus G, Insulin carrier potential for enzyme and drug therapy. Science, 223: 1304-1306, 1984.
Print C G, Loveland K L, Gibson L, Meehan T, Stylianou A, Wreford N, de Kretser D, Metcalf D, Köntgen F, Adams J M and Cory S, Apoptosis regulator Bcl-w is essential for spermatogenesis but appears otherwise redundant. Proceedings of the National Academy of Sciences of the United States of America 95: 12424-12431, 1998.
Puthalakath H, Huang D C S, O'Reilly L A, King S M and Strasser A, The proapoptotic activity of the Bcl-2 family member Bim is regulated by interaction with the dyne in motor complex. Molecular Cell 3: 287-296, 1999.
Puthalakath H, Villunger A, O'Reilly L A, Beaumont J G, Coultas L, Cheney R E, Huang D C S and Strasser A, Bmf: a pro-apoptotic BH3-only protein regulated by interaction with the myosin V actin motor complex, activated by anoikis. Science 293: 1829-1832, 2001.
Reiter Y, Brinkmann U, Jung S H, Lee B, Kasprzyk P G, King C R, Pastan I, Improved binding and antitumor activity of a recombinant anti-erbB2 immunotoxin by disulfide stabilization of the Fr fragment, J. Biol. Chem. 269: 18327-18331, 1994a.
Reiter Y, Brinkmann U, Kreitman R J, Jung S H, Lee B, Pastan I, Stabilization of the Fv fragments in recombinant immunotoxins by disulfide bonds engineered into conserved framework regions. Biochem. 33: 5451-5459, 1994b.
Reiter Y, Pai L H, Brinkmann U, Wang Q C, Pastan I, Antitumor activity and pharmacokinetics in mice of a recombinant immunotoxin containing a disulfide-stabilized Fr fragment. Cancer Res., 54: 2714-2718, 1994c.
Roberge et al., Science, 269: 202-204, 1995.
Ross A J, Waymire K G, Moss J E, Parlow A F, Skinner M K, Russell L D and MacGregor G R, Testicular degeneration in Bclw-deficient mice. Nature Genetics 18(3): 251-256, 1998.
Sambrook J et al. Molecular Cloning: A Laboratory Manual, 2^{nd} Edition, Cold Spring Harbor Laboratory Press, Plainview, N.Y., 1989.
Sattler M, Liang H, Nettesheim D, Meadows R P, Harlan J E, Eberstadt M, Yoon H S, Shuker S B, Chang B S, Minn A J, Thompson C B and Fesik S W, Structure of Bcl-x_{L}-Bak peptide complex: recognition between regulators of apoptosis. Science 275(5302):983-986, 1997.
Schimmer A D, Hedley D W, Chow S, Pham N A, Chakrabartty A, Bouchard D, Mak T W, Trus M R, Minden M D, The BH3 domain of BAO fused to the Antennapedia peptide induces apoptosis via its alpha helical structure and independent of Bcl-2. Cell Death Differ., 8 (7): 725-33, 2001.
Shangary S and Johnson D E, Peptides derived from BH3 domains of Bcl-2 family members: a comparative analysis of inhibition of Bcl-2, Bcl-x(L) and Bax oligomerization, induction of cytochrome c release, and activation of cell death. Biochemistry, 41 (30): 9485-95, 2002.
Snyder E L, Meade B R, Saenz C C, Dowdy S F, Treatment of terminal peritoneal carcinomatosis by a transducible p53-activating peptide, PloS Biology, 2, 0186-0193, 2004.
Spector M S, Desnoyers S, Hoeppner D J and Hengartner M O, Interaction between the C. elegans cell-death regulators CED-9 and CED-4. Nature 385(6617): 653-656, 1997.
Strasser, Harris A W, Bath M L and Cory S, Novel primitive lymphoid tumours induced in transgenic mice by cooperation between myc and Bcl-2. Nature 348:331-333, 1990.
Strasser A, Harris A W, Jacks T and Cory S, DNA damage can induce apoptosis in proliferating lymphoid cells via p53-independent mechanisms inhibitable by Bcl-2. Cell 79:329-339, 1994.
Strasser A, Elefanty A G, Harris A W, and Cory S, Progenitor tumours from Em-bcl-2-myc transgenic mice have lymphomyeloid differentiation potential and reveal developmental differences in cell survival. EMBO Journal 15, 3823, 1996.
Strasser A, Huang D C S and Vaux D L, The role of the Bcl-2/CED-9 gene family in cancer and general implications of defects in cell death control for tumourigenesis and resistance to chemotherapy. Biochimica et Biophysica Acta 1333:F151-F178, 1997.
Strasser A, O'Connor L and Dixit V M, Apoptosis signaling. Annual Review of Biochemistry 69: 217-245, 2000.
Supra P and Allen T M, Internalizing antibodies are necessary for improved therapeutic efficacy of antibody—targeted liposomal drugs. Cancer Res., 62 (24): 7190-4, 2002.
Suzuki M, Youle R J and Tjandra N, Structure of Bax: coregulation of dimer formation and intracellular localization. Cell 103: 645-654, 2001.
Thompson C B, Apoptosis in the pathogenesis and treatment of disease. Science 267(5203): 1456-1462, 1995.
Thornberry N A and Lazebnik Y, Caspases: enemies within. Science, 281 (5381): 1312-1316, 1998.
Thorpe P E, Wallace P M, Knowles P P, Relf M G, Brown A N. Watson G J, Knyba R E, Wawrzynczak E J, Blokey D C, New coupling reagents for the synthesis of immunotoxins containing a hindered disulfide bond with improved stability in vivo. Cancer Res., 47: 5924-5931, 1987.
Tsujimoto Y, Cossman J, Jaffe E and Croce C M, Involvement of the Bcl-2 gene in human follicular lymphoma. Science 228(4706): 1440-1443, 1985.
Uckun F N, Evans W E, Forsyth C J, Waddick K G, Ahlgren L T, Chelstrom L M, Burkhardt A, Bolen J, Meyers D E, Biotherapy of B-cell precursor leukemia by targeting genistein to CD19-associated tyrosine kinases. Science, 267 (5199): 886-898, 1995.
Vaux D L, Cory S and Adams J M, Bcl-2 gene promotes haemopoietic cell survival and cooperates with c-myc to immortalize pre-B cells. Nature 335: 440-442, 1988.
Veis D J, Sorenson C M, Shutter J R and Korsmeyer S J, Bcl-2-deficient mice demonstrate fulminant lymphoid apoptosis, polycystic kidneys, and hypopigmented hair. Cell 75: 229-240, 1993.
Verhagen A M, Ekert P G, Pakusch M, Silke J, L M C, Reid G E, Moritz R L, Simpson R J and Vaux D L, Identification of DIABLO, a mammalian protein that promotes apoptosis by binding to and antagonizing inhibitor of apoptosis (IAP) proteins. Cell 102(1): 43-53, 2000.
Vousden K H, p53; death star. Cell 103: 691-694, 2000.
Wang D, Moore S, Polyspermine-ribonuclease prepared by cross-linkage with dimethyl suberimidate. Biochemistry, 16: 2937-2941, 1977.
Wang D, Li Q, Hudson W, Berren E, Uckun F and Kersey J H, Generation and characterization of an anti-CD19 single-chain Fr immunotoxin composed of C-terminal disulfide-linked dgRTA. Bioconjug. Chem., 8 (6): 878-84, 1997.
Wang J L, Zhang Z J, Choksi S, Shan S, Lu Z, Croce C M, Alnemri E S, Korngold R, Huang Z, Cell permeable Bcl-2 binding peptides: a chemical approach to apoptosis induction in tumor cells. Cancer Res., 60 (6): 1498-502, 2000.
Wang K, Yin X-M, Chao D T, Milliman C L and Korsmeyer S J, BID: a novel BH3 domain-only death agonist. Genese and Development 10: 2859-2869, 1996.
Ward E S, Gussow D, Griffiths A D, Jones P T, Winter G, Binding activities of a repertoire of single immunoglobulin variable domains secreted from Escherichia Coli. Nature, 341: 544-546, 1989.
Webber K O, Reiter Y, Brinkmann U, Kreitman R, Pastan I, Preparation and characterisation of a disulfide-stabilized Fr fragment of the anti-Tac antibody: Comparison with its single chain analogue. Mol. Immunol. 32: 249-258, 1995.
Wei M C, Zong W X, Cheng E H, Lindsten T, Panoutsakopoulou V, Ross A J, Roth K A, MacGregor G R, Thompson C B and Korsmeyer S J, Proapoptotic BAX and BAK: a requisite gateway to mitochondrial dysfunction and death. Science 292(5517): 727-730, 2001.
Winter G and Milstein C, Man-made antibodies. Nature, 349: 293-9, 1991.
Worrell N R, Cumber A J, Parnell G D, Mirza A, Forrester J A, Ross W C, Effect of linkage variation on pharmacokinetics of ricin A chain antibody in normal rats. Anticancer Drug Design, 1: 179-188, 1986.
Wu D Y, Wallen H D and Nunez G, Interaction and regulation of subcellular localization of CED-4 and CED-9. Science 275(5303): 1126-1129, 1997.
Yang J T, Wu C S C and Martinez H G, Calculation of protein conformation by circular dichroism; Methods Enzymol., 130, 208-269, 1986.
Yang J, Liu X S, Bhalla K, Kim C N, Ibrado A M, Cai J Y, Peng T-I, Jones D P and Wang X D, Prevention of apoptosis by Bcl-2—release of cytochrome c from mitochondria blocked. Science 275(5303): 1129-1132, 1997.
Yang X, Chang H Y and Baltimore D, Essential role of CED-4 oligomerization in CED-3 activation and apoptosis. Science 281(5381): 1355-1357, 1998.
Yoshitake S, Yamada Y, Ishikawa E, Masseyeff R, Conjugation of glucose oxidase from Aspergillus niger and rabbit antibodies using N-hydroxysuccinimide ester of N-(4-carboxycyclohexylmethyl)-male-imide. Eur. J. Biochem., 101: 395-399, 1979.
Yu J, Zhang L, Hwang P M, Kinzler K W and Vogelstein B, PUMA induces the rapid apoptosis of colorectal cancer cells. Molecular Cell 7: 673-682, 2001.
Zong W X, Lindsten T, Ross A J, MacGregor G R and Thompson C B, BH3-only proteins that bind pro-survival Bcl-2 family members fail to induce apoptosis in the absence of Bax and Bak. Genes and Development 15(12): 1481-1486, 2001.
Zou H, Henzel W J, Liu X, Lutschg A and Wang X, Apaf-1, a human protein homologous to C. elegans CED-4, particpates in cytochrome c-dependent activation of Caspase-3. Cell 90: 405-4134, 1997.

1. A conjugate comprising at least one cell targeting moiety and at least one conformationally constrained peptide moiety or a pharmaceutically acceptable salt or prodrug thereof, the conformationally constrained peptide moiety comprising an amino acid sequence (I):

| | |
|---|---|
| (I) | R-(Haa₁-Saa-Xaa₁-Xaa₂)ₙ-Haa₂-Xaa₃-Xaa₄-Haa₃- |

(Saa-Naa-Xaa₅-Haa₄)ₘ-R′
wherein Haa₁, Haa₂, Haa₃ and Haa₄ are each independently an amino acid residue with a hydrophobic side chain or when n and m are both 1, one of Haa₁, Haa₂ and Haa₄ is optionally Xaa₁;
each Saa is an amino acid residue with a small side chain;
Naa is an amino acid residue with a negatively charged side chain;
Xaa₁, Xaa₂, Xaa₃, Xaa₄ and Xaa₅ are each independently an amino acid residue, Zaa₁ or Zaa₂;
R is H, an N-terminal capping group, an oligopeptide optionally capped by an N-terminal capping group or represents a linkage between the conformationally constrained peptide moiety and the cell targeting moiety;
R′ is H, a C-terminal capping group, an oligopeptide optionally capped by a C-terminal capping group or represents a linkage between the conformationally constrained peptide moiety and the cell targeting moiety; and
m and n are 0 or 1, provided that at least one of m and n is 1;
wherein a conformational constraint is provided by a linker (L) which tethers two amino acid residues, Zaa₁ and Zaa₂, in the sequence and wherein the cell targeting moiety and the conformationally constrained peptide moiety or pharmaceutically acceptable salt or prodrug thereof are coupled through R, R′ or a functionalised amino acid side chain in the amino acid sequence (I).

2. A conjugate according to claim 1, wherein in the amino acid sequence (I), all of Haa₁, Haa₂, Haa₃ and Haa₄ are amino acid residues with a hydrophobic side chain.

3. A conjugate according to claim 1, wherein in the amino acid sequence (I), Haa₁, Haa₂, Haa₃ and Haa₄ are independently selected from L-phenylalanine, L-isoleucine, L-leucine, L-valine, L-methionine and L-tyrosine.

4. A conjugate according to claim 1, wherein in the amino acid sequence (I), Haa₂ is L-leucine.

5. A conjugate according to claim 1, wherein in the amino acid sequence (I), each Saa is independently selected from glycine, L-alanine, L-serine, L-cysteine and aminoisobutyric acid.

6. A conjugate according to claim 1, wherein in the amino acid sequence (I), Naa is an L-aspartic acid or an L-glutamic acid residue.

7. A conjugate according to claim 1, wherein in the amino acid sequence (I), R is an N-terminal capping group, an oligopeptide having 1 to 10 amino acid residues selected from Xaa₁, optionally capped with an N-terminal capping group or represents a linkage between the conformationally constrained peptide moiety and the cell targeting moiety.

8. A conjugate according to claim 7, wherein R is an N-terminal capping group selected from acyl and N-succinate.

9. A conjugate according to claim 1, wherein in the amino acid sequence (I), R′ is a C-terminal capping group, an oligopeptide having 1 to 10 amino acid residues selected from Xaa₁, optionally capped with a C-terminal capping group or represents a linkage between the conformationally constrained peptide moiety and the cell targeting moiety.

10. A conjugate according to claim 9, wherein the C-terminal capping group is NH₂.

11. A conjugate according to claim 1, wherein in the amino acid sequence (I), Xaa₁, Xaa₂, Xaa₃, Xaa₄ and Xaa₅ are independently selected from L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, L-proline, L-serine, L-threonine, L-tryptophan, L-tyrosine and L-valine.

12. A conjugate according to claim 1, wherein in the amino acid sequence (I), the linker (L) tethers two non-adjacent amino acids in an i(i+7) relationship where the first end of the linker is attached to a first amino acid residue (Zaa₁) at a first position and the other end of the linker is attached to a second amino acid residue (Zaa₂) which is positioned 7 amino acids after Zaa₁.

13. A conjugate according to claim 1, wherein L is 4 to 8 atoms in length.

14. A conjugate according to claim 12, wherein in the amino acid sequence (I), Zaa₁ is located before Haa₁ at the N-terminal of the sequence and Zaa₂ is located between Haa₂ and Haa₃.

15. A conjugate according to claim 1, wherein in the amino acid sequence (I), Zaa₁ is located between Haa₁ and Haa₂ and Zaa₂ is located between Haa₃ and Haa₄.

16. A conjugate according to claim 1, wherein in the amino acid sequence (I), Zaa₁ is located between Haa₂ and Haa₃ and Zaa₂ is located after Haa₄ at the C-terminal end of the amino acid sequence.

17. A conjugate according to claim 1, wherein in the amino acid sequence (I), Zaa₁ and Zaa₂ are independently selected from L-aspartic acid, L-glutamic acid, L-lysine, L-ornithine, D-aspartic acid, D-glutamic acid, D-lysine, D-ornithine, L-β-homoaspartic acid, L-β-homoglutamic acid, L-β-homolysine, L-α-methylaspartic acid, L-α-methylglutamic acid, L-α-methyllysine, L-α-methylornithine, D-α-methylaspartic acid, D-α-methylglutamic acid, D-α-methyllysine and L-α-methylornithine.

18. A conjugate according to claim 17, wherein in the amino acid sequence (I), Zaa₁ and Zaa₂ are independently selected from L-aspartic acid, L-glutamic acid, L-lysine and L-ornithine.

19. A conjugate according to claim 18, wherein in the amino acid sequence (I), Zaa₁ and Zaa₂ are independently selected from L-aspartic acid and L-glutamic acid.

20. A conjugate according to claim 1, wherein in the amino acid sequence (I), Zaa₁ and Zaa₂ have side chains containing a carboxylic acid and the linker (L) is selected from the group consisting of —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)₂N⁺H₂(CH₂)₂NH—, —NH(CH₂)₂S(CH₂)₂NH—, —NHCH₂C(═O)NH(CH₂)₂NH—, —NH(CH₂)₂NHC(═O)CH₂NH—, —NH(CH₂)₂SS(CH₂)₂—NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(═O)NH(CH₂)₃NH—, —NH(CH2)₃NHC(═O)(CH₂)₂NH—, —NH(CH₂)₃C(═O)NH(CH₂)₂NH— and —NH(CH₂)₂NHC(—O)(CH₂)₃NH—.

21. A conjugate according to claim 20 wherein the linker is selected from the group consisting of —NH(CH₂)₅NH—, —NH(CH₂)₆NH—, —NH(CH₂)₇NH—, —NHCH₂C(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—.

22. A conjugate according to claim 20 wherein the linker is selected from the group consisting of —NH(CH₂)₅NH— and —NHCH₂C(═O)NH(CH₂)₂NH—.

23. A conjugate according to claim 1, wherein in the amino acid sequence (I), Zaa₁ and Zaa₂ have side chains containing an amino group and the linker is selected from the group consisting of —C(═O)(CH₂)₄C(═O)—, —C(═O)(CH₂)₅C(═O)—, —C(—O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂—C(═O)—, —C(═O)(CH₂)₂O(CH₂)₃C(═O)—, —C(═O)(CH₂)₂N⁺H₂(CH₂)₃C(—O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH2)₃NHC(═O)(CH₂)₂C(═O)—, —C(═O)(CH₂)₃C(═O)NH(CH₂)₂C(═O)— and —C(═O)(CH₂)₂NHC(═O)(CH₂)₃C(═O)—.

24. A conjugate according to claim 23, wherein the linker is selected from the group consisting of —C(═O)(CH₂)₅C(═O)—, —C(═O)(CH₂)₆C(═O)—, —C(═O)(CH₂)₇C(═O)—, —C(═O)CH₂C(═O)NH(CH₂)₂C(═O)—, —C(═O)(CH₂)₂NHC(═O)CH₂C(═O)—, —C(═O)(CH₂)₂O(CH₂)₃C(═O)— and —C(═O)(CH₂)₂C(═O)NH(CH₂)₂C(═O)—.

25. A conjugate according to claim 23, wherein the linker is selected from the group consisting of —C(═O)(CH₂)₅C(═O)— and —C(═O)CH₂C(═O)NH(CH₂)₂C(═O)—.

26. A conjugate according to claim 1, wherein in the amino acid sequence (I), Zaa₁ has a side chain containing an amino group and Zaa₂ has a side chain containing a carboxylic acid and the linker is selected —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂—NH—, —C(—O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(—O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NEC(═O)CH₂NH—, —C(═O)(CH₂)₂C(═O)NH(CH₂)₃NH—, —C(═O)(CH2)₃NHC(═O)(CH₂)₂NH—, —C(═O)(CH₂)₃C(═O)NH(CH₂)₂NH— and —C(═O)(CH₂)₂NHC(═O)(CH₂)₃NH—.

27. A conjugate according to claim 26 wherein the linker is selected from the group consisting of —C(═O)(CH₂)₅NH—, —C(═O)(CH₂)₆NH—, —C(═O)(CH₂)₇NH—, —C(═O)CH₂C(—O)NH(CH₂)₂NH—, —C(═O)(CH₂)₂NHC(═O)CH₂NH—, —C(═O)(CH₂)₂O(CH₂)₃NH— and —C(═O)(CH₂)₂C(═O)NH(CH₂)₂NH—.

28. A conjugate according to claim 26 wherein the linker is selected from the group consisting of —C(═O)(CH₂)₅NH— and —C(═O)CH₂C(═O)NH(CH₂)₂NH—.

29. A conjugate according to claim 1, wherein in the amino acid sequence (I), Zaa₁ has a side chain containing a carboxylic acid and Zaa₂ has a side chain containing an amino group and the linker is selected from the group consisting of —NH(CH₂)₄C(═O)—, —NH(CH₂)₅C(═O)—, —NH(CH₂)₆C(═O)—, —NH(CH₂)₇C(—O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(—O)—, —NH(CH₂)₂NHC(═O)(CH₂)₂C(═O)—, —NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)—.

30. A conjugate according to claim 29 wherein the linker is selected from the group consisting of —NH(CH₂)₅C(═O)—, —NH(CH₂)₆C(═O)—, —NH(CH₂)₇C(═O)—, —NHCH₂C(═O)NH(CH₂)₂C(═O)—, —NH(CH₂)₂NHC(═O)CH₂C(═O)—, —NH(CH₂)₂O(CH₂)₃C(═O)— and —NH(CH₂)₂C(═O)NH(CH₂)₂C(═O)—.

31. A conjugate according to claim 29 wherein the linker is selected from the group consisting of —NH(CH₂)₅C(═O)— and —NHCH₂C(═O)NH(CH₂)₂C(═O)—.

32. A conjugate according to claim 1, wherein the conformationally constrained peptide moiety or pharmaceutically acceptable salt or prodrug thereof, is selected from any one of formulae (II) to (VI): 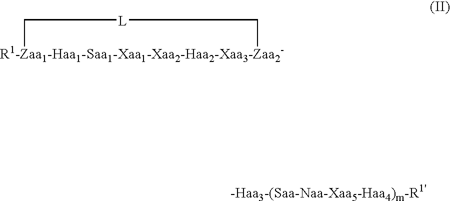
wherein Haa₁, Haa₂, Haa₃, Haa₄, Xaa₁, Xaa₂, Xaa₃, Xaa₅, Saa, Naa and L are as defined above for formula (I), m is 0 or 1, R¹ and R^{1′} are as defined above for R and R′ in formula (I), Zaa₁-L-Zaa₂ represents two amino acid residues with their side chains bridged by a linker L, and the cell targeting moiety is coupled to the peptide moiety through R¹, R^{1′} or through a functionalized amino acid side chain in the peptide; 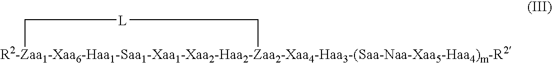
wherein Haa₁, Haa₂, Haa₃, Haa₄, Xaa₁, Xaa₂, Xaa₄, Xaa₅, Saa, Naa and L are as defined above for formula (I), Xaa₆ is an amino acid residue as defined for Xaa₁ above; m is 0 or 1, R² and R^{2′} are as defined above for R and R′ in formula (I), Zaa₁-L-Zaa₂ represents two amino acid residues with their side chains bridged by a linker L, and the cell targeting moiety is coupled to the peptide moiety through R², R^{2′} or through a functionalized amino acid side chain in the peptide; 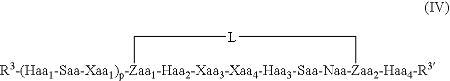
wherein Haa₁, Haa₂, Haa₃, Haa₄, Xaa₁, Xaa₃, Xaa₄, Saa, Naa and L are as defined above for formula (I), p is 0 or 1, R³ and R^{3′} are as defined above for R and R′ in formula (I), Zaa₁-L-Zaa₂ represents two amino acid residues with their side chains bridged by a linker L, and the cell targeting moiety is coupled to the peptide moiety through R³, R^{3′} or through a functionalized amino acid side chain in the peptide; 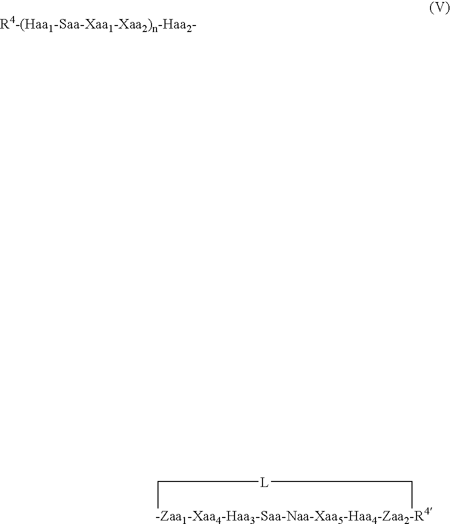
wherein Haa₁, Haa₂, Haa₃, Haa₄, Xaa₁, Xaa₂, Xaa₄, Xaa₅, Saa, Naa and L are as defined above in formula (I), n is 0 or 1, R⁴ and R^{4′} are as defined above for R and R′ in formula (I), Zaa₁-L-Zaa₂ represents two amino acid residues with their side chains bridged by a linker L, and the cell targeting moiety is coupled to the peptide moiety through R⁴, R^{4′} or through a functionalized amino acid side chain in the peptide; and 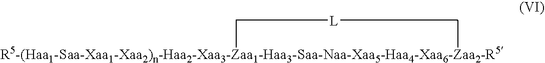
wherein Haa₁, Haa₂, Haa₃, Haa₄, Xaa₁, Xaa₂, Xaa₃, Xaa₅, Saa, Naa and L are as defined above for formula (I), Xaa₆ is an amino acid residue as defined for Xaa₁ above; n is 0 or 1, R⁵ and R^{5′} are as defined above for R and R′ in formula (I), Zaa₁-L-Zaa₂ represents two amino acid residues with their side chains bridged by a linker L, and the cell targeting moiety is coupled to the peptide moiety through R⁵, R^{5′} or through a functionalized amino acid side chain in the peptide;
or a pharmaceutically acceptable salt or prodrug thereof.

33. A conjugate according to claim 32 comprising a conformationally constrained peptide moiety or pharmaceutically acceptable salt or prodrug thereof having structural formula (VII): 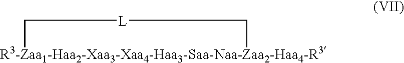
wherein Zaa₁, Haa₂, Xaa₃, Xaa₄, Haa₃, Saa, Naa, Zaa₂, Haa₄, R³, R^{3′} and L are defined above in formula (IV), and the cell targeting moiety is coupled to the peptide moiety through R³, R^{3′} or a functionalized amino acid side chain in the peptide.

34. A conjugate according to claim 1 comprising a conformationally constrained peptide moiety or pharmaceutically acceptable salt or prodrug thereof having structural formula (VIII): 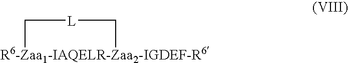
wherein R⁶ is Acetyl or represents a linkage with the cell targeting moiety;
R^{6′} is NH₂ or represents a linkage with the cell targeting moiety; and
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH— and —NH(CH₂)₂NHC(═O)(CH₂)₂NH—; or
where Zaa₁ and Zaa₂ are selected from L-lysine and ornithine; and
L is selected from —C(═O)(CH₂)₄C(—O)—, —C(═O)(CH₂)₅C(—O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)— and —C(═O)(CH₂)₂NHC(═O)(CH₂)₂C(—O)—; or
where Zaa₁ is selected from L-aspartic acid, L-glutamic acid and Zaa₂ is selected from L-lysine and ornithine; and
L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(—O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)— and —NH(CH₂)₂NHC(═O)(CH₂)₂C(═O)—; or
where Zaa₁ is selected from L-lysine and ornithine and Zaa₂ is selected from L-aspartic acid, L-glutamic acid; and
L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH— and —C(═O)(CH₂)₂NHC(—O)(CH₂)₂NH—;
and where the cell targeting moiety and the peptide moiety are coupled through R⁶, R^{6′} or a functionalized amino acid side chain in the peptide.

35. A conjugate according to claim 1, comprising a conformationally constrained peptide moiety or pharmaceutically acceptable salt or prodrug thereof having structural formula (IX): 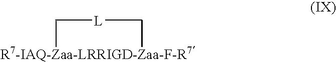
wherein R⁷ is Acetyl or represents a linkage with the cell targeting moiety;
R^{7′} is NH₂ or represents a linkage with the cell targeting moiety; and
where Zaa₁ and Zaa₂ are selected from L-aspartic acid, L-glutamic acid; and
L is selected from —NH(CH₂)₄NH—, —NH(CH₂)₅NH—, —NH(CH₂)₆NH—, —NH(CH₂)₇NH—, —NH(CH₂)₂O(CH₂)₂NH—, —NH(CH₂)N⁺H₂(CH₂)₂NH—, —NH(CH₂)S(CH₂)₂NH—, —NHCH₂C(═O)NH(CH₂)₂NH—, —NH(CH₂)₂NHC(═O)CH₂NH—, —NH(CH₂)₂SS(CH₂)₂NH—, —NH(CH₂)₂O(CH₂)₃NH—, —NH(CH₂)₂N⁺H₂(CH₂)₃NH—, —NH(CH₂)₂S(CH₂)₃NH—, —NH(CH₂)₂C(═O)NH(CH₂)₂NH—, —NH(CH₂)₂NHC(═O)(CH₂)₂NH—, —NHCH₂C(═O)NH(CH₂)₃NH—, —NH(CH₂)₃NHC(═O)CH₂NH—, —NHCH₂C(═O)NH(CH₂)₄NH—, —NH(CH₂)₄NHC(═O)CH₂NH—, —NH(CH₂)₂C(—O)NH(CH₂)₃NH—, —NH(CH₂)₃NHC(═O)(CH₂)₂NH—, —NH(CH₂)₃C(═O)NH(CH₂)₂NH— and —NH(CH₂)₂NHC(═O)(CH₂)₃NH—; or
where Zaa₁ and Zaa₂ are selected from L-lysine and ornithine; and
L is selected from —C(═O)(CH₂)₄C(═O)—, —C(═O)(CH₂)₅C(═O)—, —C(═O)(CH₂)₆C(═O)—, —C(═O)(CH₂)₇C(═O)—, —C(═O)(CH₂)₂O(CH₂)₂C(═O)—, —C(═O)(CH₂)N⁺H₂(CH₂)₂C(═O)—, —C(═O)(CH₂)S(CH₂)₂C(═O)—, —C(═O)CH₂C(═O)NH(CH₂)₂C(═O)—, —C(═O)(CH₂)₂NHC(═O)CH₂C(═O)—, —C(═O)(CH₂)₂SS(CH₂)₂C(═O)—, —C(═O)(CH₂)₂O(CH₂)₃C(═O)—, —C(═O)(CH₂)₂N⁺H₂(CH₂)₃C(═O)—, —C(═O)(CH₂)₂S(CH₂)₃C(═O)—, —C(═O)(CH₂)₂C(═O)NH(CH₂)₂C(═O)—, —C(═O)(CH₂)₂NHC(═O)(CH₂)₂C(═O)—, —C(═O)CH₂C(═O)NH(CH₂)₃C(═O)—, —C(═O)(CH₂)₃NHC(═O)CH₂C(═O)—, —C(═O)CH₂C(═O)NH(CH₂)₄C(═O)—, —C(═O)(CH₂)₄NHC(═O)CH₂C(═O)—, —C(═O)(CH₂)₂C(═O)NH(CH₂)₃C(═O)—, —C(═O)(CH₂)₃NHC(═O)(CH₂)₂C(═O)—, —C(═O)(CH₂)₃C(═O)NH(CH₂)₂C(═O)— and —C(═O)(CH₂)₂NHC(—O)(CH₂)₃C(═O)—; or
where Zaa₁ is selected from L-aspartic acid, L-glutamic acid and Zaa₂ is selected from L-lysine and ornithine; and
L is selected from —NH(CH₂)₄C(═O)—, —NH(CH₂)₅C(═O)—, —NH(CH₂)₆C(═O)—, —NH(CH₂)₇C(═O)—, —NH(CH₂)₂O(CH₂)₂C(═O)—, —NH(CH₂)N⁺H₂(CH₂)₂C(═O)—, —NH(CH₂)S(CH₂)₂C(═O)—, —NHCH₂C(═O)NH(CH₂)₂C(═O)—, —NH(CH₂)₂NHC(═O)CH₂C(═O)—, —NH(CH₂)₂SS(CH₂)₂C(═O)—, —NH(CH₂)₂O(CH₂)₃C(═O)—, —NH(CH₂)₂N⁺H₂(CH₂)₃C(═O)—, —NH(CH₂)₂S(CH₂)₃C(═O)—, —NH(CH₂)₂C(═O)NH(CH₂)₂C(═O)—, —NH(CH₂)₂NHC(═O)(CH₂)₂C(═O)—, —NHCH₂C(═O)NH(CH₂)₃C(═O)—, —NH(CH₂)₃NHC(═O)CH₂C(═O)—, —NHCH₂C(═O)NH(CH₂)₄C(═O)—, —NH(CH₂)₄NHC(═O)CH₂C(═O)—, —NH(CH₂)₂C(═O)NH(CH₂)₃C(═O)—, —NH(CH₂)₃NHC(═O)(CH₂)₂C(═O)—, —NH(CH₂)₃C(═O)NH(CH₂)₂C(═O)— and —NH(CH₂)₂NHC(═O)(CH₂)₃C(═O)—; or
where Zaa₁ is selected from L-lysine and ornithine and Zaa₂ is selected from L-aspartic acid, L-glutamic acid; and
L is selected from —C(═O)(CH₂)₄NH—, —C(═O)(CH₂)₅NH—, —C(═O)(CH₂)₆NH—, —C(═O)(CH₂)₇NH—, —C(═O)(CH₂)₂O(CH₂)₂NH—, —C(═O)(CH₂)N⁺H₂(CH₂)₂NH—, —C(═O)(CH₂)S(CH₂)₂NH—, —C(═O)CH₂C(═O)NH(CH₂)₂NH—, —C(═O)(CH₂)₂NHC(═O)CH₂NH—, —C(═O)(CH₂)₂SS(CH₂)₂NH—, —C(═O)(CH₂)₂O(CH₂)₃NH—, —C(═O)(CH₂)₂N⁺H₂(CH₂)₃NH—, —C(═O)(CH₂)₂S(CH₂)₃NH—, —C(═O)(CH₂)₂C(═O)NH(CH₂)₂NH—, —C(═O)(CH₂)₂NHC(═O)(CH₂)₂NH—, —C(═O)CH₂C(═O)NH(CH₂)₃NH—, —C(═O)(CH₂)₃NHC(═O)CH₂NH—, —C(═O)CH₂C(═O)NH(CH₂)₄NH—, —C(═O)(CH₂)₄NHC(═O)CH₂NH—, —C(═O)(CH₂)₂C(═O)NH(CH₂)₃NH—, —C(═O)(CH₂)₃NHC(═O)(CH₂)₂NH—, —C(═O)(CH₂)₃C(═O)NH(CH₂)₂NH— and —C(═O)(CH₂)₂NHC(═O)(CH₂)₃NH—,
and where the cell targeting moiety and the peptide moiety are coupled through R⁷, R^{7′} or a functionalized amino acid side chain in the peptide.

36. A conjugate according to claim 1 comprising a conformationally constrained peptide moiety or pharmaceutically acceptable salt or prodrug thereof selected from the group consisting of: 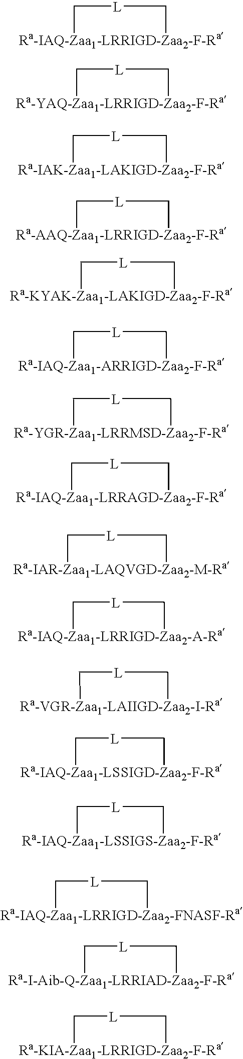
where R^{a} is acetyl or represents the linkage with the cell targeting moiety and R^{a′} is NH₂ or represents the linkage with the cell targeting moiety. In each case, the cell targeting moiety may be coupled to the peptide through R^{a}, R^{a′} or a functionalized amino acid side chain in the peptide, and
wherein Zaa₁ and Zaa₂ are as defined in claim 17 and L is a linker which tethers Zaa₁ and Zaa₂.

37. A conjugate according to claim 36, wherein Zaa₁ and Zaa₂ are independently selected from L-aspartic acid and L-glutamic acid and L is selected from the group consisting of —NH(CH₂)₅NH—, —NH(CH₂)₆NH—, —NH(CH₂)₇NH—, —NHCH₂(═O)NH(CH₂)₂NH—, —NH(CH₂)₂NHC(═O)CH₂NH—, —NH(CH₂)₂O(CH₂)₃NH— and —NH(CH₂)₂C(═O)NH(CH₂)₂NH—.

38. A conjugate according to claim 37 wherein L is selected from the group consisting of —NH(CH₂)₅NH— and —NHCH₂C(═O)NH(CH₂)₂NH—.

39. A conjugate according to claim 1, comprising a conformationally constrained peptide moiety or pharmaceutically acceptable salt or prodrug thereof selected from the group consisting of: 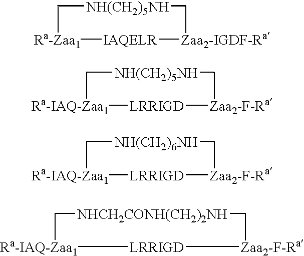
where R^{a} is Acetyl or represents a linkage to the cell targeting moiety, R^{a′} is NH₂ or represents a linkage to the cell targeting moiety and where the cell targeting moiety is coupled to the peptide through R^{a}, R^{a′} or a functionalized amino acid side chain in the peptide, and
wherein Zaa₁ and Zaa₂ are independently selected from L-aspartic acid and L-glutamic acid.

40. A conjugate according to claim 39 wherein Zaa₁ and Zaa₂ are both L-glutamic acid.

41. A conjugate according to claim 1, wherein the cell targeting moiety is an antigen-binding molecule.

42. A conjugate according to claim 1, wherein the cell targeting moiety is a hormone, a cytokine or an antibody.

43. A conjugate according to claim 42, wherein the hormone is luteinising hormone-releasing hormone.

44. A conjugate according to claim 42, wherein the cytokine is selected from VEGF and EGF.

45. A conjugate according to claim 42, wherein the antibody is selected from CD19, CD20, CD22, CD79a, CD2, CD3, CD7, CD5, CD13, CD33, CD138 antibodies and antibodies targeting Erb1, Erb2, Erb3 or Erb4 receptors.

46. A conjugate according to claim 45, wherein the antibody is selected from CD19, CD20, CD22 and CD79a antibodies.

47. A pharmaceutical composition comprising a conjugate according to claim 1, together with one or more pharmaceutically acceptable carriers and optionally, other therapeutic and/or prophylactic ingredients.

48. A method of regulating the death of a cell, comprising contacting the cell with an effective amount of a conjugate according to claim 1.

49. A method of inducing apoptosis in unwanted or damaged cells comprising contacting said damaged or unwanted cells with an effective amount of a conjugate according to claim 1.

50. A method of treatment and/or prophylaxis of a pro-survival Bcl-2 family member-mediated disease or condition, in a mammal, comprising administering to said mammal an effective amount of a conjugate according to claim 1.

51. A method according to claim 50 wherein the disease or condition is an inflammatory condition, a cancer or an autoimmune disorder.

52. A method of treatment and/or prophylaxis of a disease or condition characterised by the inappropriate persistence or proliferation of unwanted or damaged cells in a mammal, comprising administering to said mammal an effective amount of a conjugate according to claim 1.

53. A method according to claim 52, wherein the unwanted or damaged cells are B cells and the cell targeting moiety of the conjugate is selected from CD19, CD20, CD22 and CD79a antibodies.

54. A method according to claim 53, wherein the disease or condition is selected from B cell non-Hodgkins Lymphoma, B cell acute lymphoblastic leukemia, rheumatoid arthritis, systemic Lupus erythematosis and related arthropathies.

55. A method according to claim 52, wherein the unwanted or damaged cells are T cells and the cell targeting moiety of the conjugate is selected from CD2, CD3, CD7 and CD5.

56. A method according to claim 55, wherein the disease or condition is selected from T cell acute lymphoblastic leukemia, T cell non-Hodgkins lymphoma and Graft vs Host disease.

57. A method according to claim 52, wherein the unwanted or damaged cells are myeloid cells and the cell targeting moiety of the conjugate is selected from CD13, and CD33.

58. A method according to claim 57, wherein the disease or condition is selected from acute myelogenous leukemia, chronic myelogenous leukemia and chronic myelomonocytic leukemia.

59. A method according to claim 52, wherein the unwanted or damaged cells are plasma cells and the cell targeting moiety of the conjugate is CD138.

60. A method according to claim 59, wherein the disease or condition is multiple myeloma.

61. A method according to claim 52, wherein the unwanted or damaged cells are cancer cells and the cell targeting moiety of the conjugate is luteinizing hormone-releasing hormone.

62. A method according to claim 61, wherein the disease or condition is selected from ovarian cancer, breast cancer and prostate cancer.

63. Use of a conjugate according to claim 1, in the manufacture of a medicament for regulating the death of a cell, for inducing apoptosis in unwanted or damaged cells, for the treatment and/or prophylaxis of a pro-survival Bcl-2 family member-mediated disease or condition, or for the treatment and/or prophylaxis of a disease or condition characterised by the inappropriate persistence or proliferation of unwanted or damaged cells.

64. A method of preparing a conformationally constrained peptide comprising the steps of:
(i) reacting a linker containing a first functional group and a second functional group with a reactive group on an amino acid side chain so that the first functional group of the linker is covalently coupled with the reactive group of the amino acid side chain;
(ii) protecting the second functional group of the linker if required;
(iii) incorporating the amino acid from (i) or (ii) into a peptide, said peptide comprising a second amino acid having a reactive side chain capable of covalently coupling with the second functional group of the linker;
(iv) deprotecting the second functional group of the linker if required; and
(v) reacting the second functional group of the linker with the reactive side chain of the second amino acid.

65. A method according to claim 64 comprising the steps of:
(i) reacting a linker having one amino group and one optionally protected amino group or one amino group and one optionally protected carboxylic acid group, with an amino acid having a side chain comprising a carboxylic acid so that the linker and the amino acid side chain are coupled by an amide bond;
(ii) incorporating the amino acid from (i) into a peptide, said peptide comprising a second amino acid residue having a side chain capable of reacting with the uncoupled amino group or carboxylic acid group of the linker;
(iii) deprotecting the amino group or carboxylic acid group of the linker if required; and
(iv) reacting the second amino acid side chain with the amino group or carboxylic acid group of the linker to form an amide bond.

66. A method according to claim 64 comprising the steps of:
(i) reacting a linker having one carboxylic acid group and one optionally protected carboxylic acid group or one carboxylic acid group and one optionally protected amino group, with an amino acid having a side chain comprising an amino group so that the linker and the amino acid side chain are coupled by an amide bond;
(ii) incorporating the amino acid from (i) into a peptide, said peptide comprising a second amino acid residue having a side chain capable of reacting with the uncoupled amino group or carboxylic acid group of the linker;
(iii) deprotecting the amino group or carboxylic acid group of the linker; and
(iv) reacting the second amino acid side chain with the carboxylic acid group or amino group of the linker to form an amide bond.
